(19) United States
(12) Patent Application Publication
(10) Pub. No.: US 2002/0086300 A1
(43) Pub. Date: Jul. 4, 2002

(54) Novel signal transduction molecules

(76) Inventors: Jon Elliot Adler, San Diego, CA (US); Shawn M. O'Connell, Encinitas, CA (US)

Correspondence Address: Pillsbury Winthrop LLP, Intellectual Property Group, East Tower, Ninth Floor, 1100 New York Avenue, N.W., Washington, DC, 20005-3918, US

(21) Appl. No.: 09/826,001

(22) Filed: Apr. 5, 2001

Related U.S. Application Data
(60) Provisional application No. 60/195,534, filed on Apr. 7, 2000.
(60) Provisional application No. 60/259,514, filed on Jan. 4, 2001.

(51) Int. Cl. C12Q 1/68; C07H 21/04; C12N 5/06; C07K 14/705

(52) U.S. Cl. 435/6; 435/325; 435/320.1; 536/23.5; 530/350

(57) ABSTRACT

Newly identified signal transduction polypeptides active in taste signal transduction, and the genes encoding said polypeptides are described. Specifically, sensory proteins referred to as REPEATER, LUNCH, and 165-015 polypeptides, are described, and genes encoding the same are described, along with methods for isolating such genes and for expressing polypeptides and analyzing signal transduction interactions. Methods for identifying novel molecules or combinations of molecules that are involved in taste signal transduction in a mammal are also described, which utilize at least one of the identified signal transduction polypeptides.

### CROSS REFERENCE TO RELATED APPLICATIONS

This application claims priority to U.S. Ser. No. 60/195,534, filed Apr. 7, 2000, and U.S. Ser. No. 60/259,514, filed Jan. 4, 2001, both of which are herein incorporated by reference in their entireties.

### FIELD OF THE INVENTION

The invention relates to a newly identified proteins involved in signal transduction, particularly to a newly identified proteins that are expressed in taste receptor cells, and to the genes encoding said proteins.

### BACKGROUND OF THE INVENTION

The invention relates to newly identified taste-related signal transduction polypeptides, to families of such polypeptides, and to the genes and cDNA encoding such polypeptides. More particularly, the invention relates to newly identified taste cell signal transduction polypeptides active in taste signaling, to families of such polypeptides, to the genes and cDNA such polypeptides, and to methods of using such polypeptides, genes, and cDNA in the analysis and discovery of taste modulators.

### DESCRIPTION OF THE RELATED ART

The taste system provides sensory information about the chemical composition of the external world. Taste transduction is one of the most sophisticated forms of chemical-triggered sensation in animals, and is found throughout the animal kingdom, from simple metazoans to the most complex of vertebrates. Mammals are believed to have five basic taste modalities: sweet, bitter, sour, salty, and umami (the taste of monosodium glutamate).

Each taste modality is believed to be mediated by distinct transduction pathways. These pathways are believed to be mediated by receptors, e.g., metabotropic or ionotropic receptors, expressed in subsets of taste receptor cells. For instance, some tastes are believed to be mediated by G Protein-Coupled Receptors, while other tastes are believed to be mediated by channel proteins (see, e.g., Kawamura et al, Introduction to Umami. A Basic Taste (1987); Kinnamon et al., Ann. Rev. Physiol., 54:715-31 (1992); Lindemann, Physiol. Rev., 76:718-66 (1996); Stewart et al., Am. J. Physiol., 272:1-26(1997)).

In mammals, taste receptor cells are assembled into taste buds that are distributed into different papillae in the tongue epithelium. Circumvallate papillae, found at the very back of the tongue, contain hundreds to thousands of taste buds. By contrast, foliate papillae, localized to the posterior lateral edge of the tongue, contain dozens to hundreds of taste buds. Further, fungiform papillae, located at the front of the tongue, contain only a small number of taste buds.

Each taste bud, depending on the species, contains 50-150 cells, including precursor cells, support cells, and taste receptor cells. See, e.g., Lindemann, Physiol. Rev., 76:718-66 (1996). Receptor cells are innervated at their base by afferent nerve endings that transmit information to the taste centers of the cortex through synapses in the brain stem and thalamus. Elucidating the mechanisms of taste cell signaling and information processing is important to understanding the function, regulation, and perception of the sense of taste.

Numerous physiological studies in animals have shown that taste receptor cells may selectively respond to different chemical stimuli (see, e.g., Akabas et al., Science, 242:1047-50 (1988); Gilbertson et al., J. Gen. Physiol., 100:803-24 (1992); Bernhardt et al., J. Physiol., 490:325-36 (1996); Cummings et al., J. Neurophysiol., 75:1256-63 (1996)). More particularly, cells that express taste receptors, when exposed to certain chemical stimuli, elicit taste sensation by depolarizing to generate an action potential. The action potential is believed to trigger the release of neurotransmitters at gustatory afferent neuron synapses, thereby initiating signaling along neuronal pathways that mediate taste perception (see, e.g., Roper, Ann. Rev. Neurosci., 12:329-53 (1989)). Nonetheless, at present, the means by which taste sensations are elicited remains poorly understood (see, e.g., Margolskee, BioEssays, 15:645-50 (1993); Avenet et al., J. Membrane Biol., 112:1-8 (1989)).

As described above, taste receptors specifically recognize molecules that elicit specific taste sensation. These molecules are also referred to herein as “tastants.” Many taste receptors belong to the 7-transmembrane receptor superfamily (Hoon et al., Cell 96:451 (1999); Adler et al., Cell 100:693 (2000)), which are also known as G Protein-Coupled Receptors (GPCRs). G Protein-Coupled Receptors control many physiological functions, such as endocrine function, exocrine function, heart rate, lipolysis, and carbohydrate metabolism. The biochemical analysis and molecular cloning of a number of such receptors has revealed many basic principles regarding the function of these receptors.

For example, U.S. Pat. No. 5,691,188 describes how upon a ligand binding to a GPCR, the receptor presumably undergoes a conformational change leading to activation of the G Protein. G Proteins are comprised of three subunits: a guanyl nucleotide binding α subunit, a β subunit, and a γ subunit. G Proteins cycle between two forms, depending on whether GDP or GTP is bound to the cc subunit. When GDP is bound, the G Protein exists as a heterotrimer: the Gαβγ complex. When GTP is bound, the α subunit dissociates from the heterotrimer, leaving a Gβγ complex. When a Gαβγ complex operatively associates with an activated G Protein-Coupled Receptor in a cell membrane, the rate of exchange of GTP for bound GDP is increased and the rate of dissociation of the bound Gα subunit from the Gαβγ complex increases. The free Gα subunit and Gβγ complex are thus capable of transmitting a signal to downstream elements of a variety of signal transduction pathways. These events form the basis for a multiplicity of different cell signaling phenomena, including for example the signaling phenomena that are identified as neurological sensory perceptions such as taste and/or smell.

Although much is known about the psychophysics and physiology of taste cell function, very little is known about the signal transduction molecules and pathways that mediate its sensory signaling response. The identification and isolation of novel taste-related signal transduction molecules (TSTPs) could allow for new methods of chemical and genetic modulation of taste transduction pathways. For example, the availability of TSTPs could permit screening for high affinity agonists, antagonists, inverse agonists, and modulators of taste activity. Such taste modulating compounds could be useful in the pharmaceutical and food industries to improve the taste of a variety of consumer products, or to block undesirable tastes.

### SUMMARY OF THE INVENTION

The invention relates to newly identified polypeptides active in taste signal transduction, and to the genes encoding said polypeptides. In particular, the invention provides novel families of taste-related signal transduction polypeptides (TSTPs), designated REPEATER, LUNCH, and 165-015. In part, the invention provides mammalian TSTPs based on identification of orthologs in rat, mouse (murine), and human genomes.

Toward that end, it is an object of the invention to provide new TSTPs. It is another object of the invention to provide fragments and variants of such TSTPs which retain signal transduction activity. It is yet another object of the invention to provide nucleic acid sequences or molecules that encode such TSTPs, fragments, and variants thereof.

It is still another object of the invention to provide expression vectors which include nucleic acid sequences that encode such TSTPs, fragments, or variants thereof, which are operably linked to at least one regulatory sequence such as a promoter, enhancer, and other sequences involved in positive and negative gene transcription and/or translation.

It is still another object of the invention to provide human or non-human cells which functionally express at least one of such TSTPs, fragments or variants thereof. Such cells may be used to study the mechanism by which the TSTPs of the invention are involved in signal transduction, and to screen for compounds which modulate, inhibit or enhance sensory signals.

It is still another object of the invention to provide fusion proteins or which include at least a fragment of at least one of the TSTPs disclosed herein. Such fusion proteins may be used to study protein function or localization in the cell, screen for compounds or drugs which activate the expression of genes involved in signal transduction, screen for cells which express different paralogs of the genes described herein, etc.

It is another object of the invention to provide an isolated nucleic acid molecule encoding a TSTP comprising a nucleic acid sequence that is at least 75%, preferably 85%, 90%, 95%, 96%, 97%, 98%, or 99% identical to a nucleic acid sequence selected from the group consisting of: SEQ ID NOS: 4, 6, 10, 12, 14, 21, 23, and conservatively modified variants thereof.

It is a further object of the invention to provide an isolated nucleic acid molecule comprising a nucleic acid sequence that encodes a TSTP having an amino acid sequence at least 75%, preferably 85%, 90%, 95%, 96%, 97%, 98%, or 99% identical to an amino acid sequence selected from the group consisting of: SEQ ID NOS: 1, 2, 3, 5, 7, 9, 11, 12, 15, 16, 17, 18, 19, 20, 22, 24, and conservatively modified variants thereof, wherein the polypeptide is at least 20, preferably at least 40, 60, 80, 100, 150, 200, or 250 amino acids in length. Optionally, the polypeptide can be an antigenic fragment which binds to an anti-REPEATER or anti-LUNCH, or anti-165 antibody.

It is still a further object of the invention to provide an isolated TSTP comprising a variant of a polypeptide having an amino acid sequence selected from the group consisting of: SEQ ID NOS: 1, 2, 3, 5, 7, 9, 11, 12, 15, 16, 17, 18, 19, 20, 22, 24, wherein there is a variation in at most 10, preferably 5, 4, 3, 2, or 1 amino acid residues. Such variations may be conservative substitutions which do not change the function of the TSTP, or deletions or insertions which decrease or enhance activity.

It is still another object of the invention to provide methods of screening for one or more compounds which modulate taste transduction using the TSTPs of the invention, and particularly compounds that modulate taste transduction in cells expressing said TSTPs. The agonists, antagonists, inhibitors, modulators, activators, etc. of taste transduction identified according to the methods of the invention are also encompassed by the present invention.

### DETAILED DESCRIPTION OF THE INVENTION

The invention thus provides isolated nucleic acid molecules encoding taste-related signal transduction polypeptides (TSTPs), and the polypeptides they encode. These nucleic acid molecules and the polypeptides that they encode are members of the REPEATER, LUNCH, and 165-015 families of TSTPs, and are collectively referred to herein as TSTPs.

More particularly, the recent identification of the rat REPEATER, LUNCH, and 165-015 genes, prompted the search for, and identification of related genes in public nucleotide sequence databases. For instance, the rat 165-015A gene has been identified as being selectively expressed in certain rat taste cells. Further, rat REPEATER gene has been found to be selectively expressed by all cells of taste buds of the posterior tongue, and anti-REPEATER antibodies have been used to demonstrate that the rat REPEATER protein is localized to and secreted at the taste pore. Likewise, the rat LUNCH gene has been found to be selectively expressed by a subset of cells of taste buds of the posterior tongue, and anti-LUNCH antibodies have been used to demonstrate that the rat LUNCH protein is localized to the taste pore. Six splice variants of rat LUNCH have also been identified. The present invention relates to newly identified members of these families of TSTPs. Further information regarding these families can be found in WO 00/06719, which is herein incorporated by reference in its entirety. WO 00/06719 refers to REPEATER genes as TCP #1, to LUNCH genes as TCP #2, and to 165-015 genes as TCP #3.

In one aspect of the invention, a human ortholog of rl65-015A is described herein, as well as five additional related human genes (paralogs). The human 165-015 A, B, and F genes are linked, and the E and D genes are linked. The letter designations, such as the A of rl65-015A and hl65-015A, are used to match orthologs. Also identified herein are segments of the rat 165-015B and 165-015C genes; the full length mouse 165-015C gene; a segment of the mouse 165-015D gene; segments of the pig and cow 165-015B genes; and of a gene of distantly-related C. elegans. Alignments of the predicted gene product sequences for the disclosed gene family in combination with cDNA-based experiments indicate that the database annotation for the sequence of the C. elegans gene product is incorrect because of a splice junction misalignment.

In another aspect of the invention, a segment of the mouse REPEATER gene was cloned from genomic DNA (contained in plasmid SAV264), and used to generate [S-35]-labeled riboprobes for in situ hybridization experiments. Using these riboprobes, it was discovered that in addition to robust expression in posterior taste buds, the REPEATER gene is strongly expressed throughout the dorsal lingual and palate epithelium, as well as the gastrointestinal epithelium.

Further, in another aspect of the invention, the human REPEATER gene sequence was identified from three genomic intervals (accessions AC006163, AP000510, and AB023060) (presented below in the Examples as SEQ ID NO: 21). The human REPEATER gene contains a single, coding sequence-interrupting intron. Its conceptual translation (SEQ ID NO: 22) is approximately 45% identical over the first 270 amino acids to rat REPEATER protein. The alignment beyond 270 amino acids degrades because of divergence in the number of 11-12 residue repeats that make up the C-terminal halves of these proteins. This gene was independently identified as a new, functionally undefined locus in the HLA class I region on chromosome 6, and a full length cDNA was cloned from keratinocytes (accession AB031481; see Hum. Mol. Genet., 8, 2165-70 (1999)).

In yet another aspect of the invention, the human LUNCH gene was identified in the chromosome 15 genomic interval corresponding to accession AC024552. The low information content of LUNCH protein, which is highly enriched in proline and serine residues, and multiple coding exons of the human LUNCH-like gene complicate exon calling and coding sequence prediction. Consequently, only coding sequence derived from 8 exons and its conceptual translation are presented herein as SEQ ID NOS: 23 and 24. The human LUNCH sequence corresponds, with 61% identity, to residues 393 to 683 of the long isoform of rat LUNCH. It is believed that the full length human LUNCH sequence will correspond with the same or similar identity to the full length rat LUNCH sequence disclosed in WO 00/06719 (TCP #2). As such, the full length human LUNCH polypeptide and the gene encoding it are also envisioned to be within the scope of the present invention.

As described above, these genes are predicted to encode taste-related polypeptides exhibiting signal transduction functions, and are believed to be components of a signal transduction cascade. They may, for example, form ion channels or they may regulate receptor trafficking or function. The different members of the TSTP families may have specialized to function in different tissues. Accordingly, the TSTPs disclosed herein are believed to be involved in taste signaling, and taste perception may be modulated by molecules identified using such TSTPs.

Modulators identified using the TSTPs disclosed herein may modulate taste receptor cell signaling, thereby altering taste perception. Further, such modulators may modulate the signal transduction function of paralogs of TSTPs in other cell types in the body. Thus, in part, the present invention is directed to TSTPs which can be used to identify variant proteins or to identify modulatory compounds that may be used to inhibit or enhance taste cell signal transduction. The term modulation is used herein to refer to the capacity to either enhance or inhibit a biological activity of a TSTP of the present invention, to the capacity to either enhance or inhibit the biological activity of a taste receptor cell, or to the capacity to either enhance or inhibit a functional property of a nucleic acid coding region of a nucleic acid molecule encoding a TSTP of the present invention.

The present invention encompasses the specific TSTPs and genes disclosed herein, as well as orthologs and paralogs in other mammalian species. In one embodiment, the present invention is directed to an isolated nucleic acid molecule comprising a nucleic acid sequence selected from the group consisting of (i) a nucleic acid sequence having at least 75%, 80%, 85%, 90%, 95%, 96%, 97%, 98%, or 99% identity to a sequence selected from SEQ ID NOS: 4, 6, 8, 10, 12, 14, 21, 23, and conservatively modified variants thereof; (ii) a nucleic acid sequence coding for a TSTP comprising an amino acid sequence having at least 75%, 80%, 85%, 90%, 95%, 96%, 97%, 98%, or 99% identity to a sequence selected from SEQ ID NOS: 1, 2, 3, 5, 7, 9, 11, 12, 15, 16, 17, 18, 19, 20, 22, 24, and conservatively modified variants thereof; (iii) a variant of a nucleotide sequence of SEQ ID NOS: 4, 6, 8, 10, 12, 14, 21, or 23, containing at least one conservative substitution in a region coding for a TSTP; and (iv) a variant of a nucleotide sequence encoding a TSTP having an amino acid sequence of SEQ ID NOS: 1, 2, 3, 5, 7, 9, 11, 12, 15, 16, 17, 18, 19, 20, 22, or 24, containing at least one conservative substitution in a TSTP coding region.

As used herein, the term “isolated,” when referring to a nucleic acid or polypeptide refers to a state of purification or concentration different than that which occurs naturally in a mammalian body. Any degree of purification or concentration greater than that which occurs naturally in the body, including (1) the purification from other naturally-occurring associated structures or compounds, or (2) the association with structures or compounds to which it is not normally associated in the body are within the meaning of “isolated” as used herein. The nucleic acids or polypeptides described herein may be isolated or otherwise associated with structures or compounds to which they are not normally associated in nature, according to a variety of methods and processed known to those of skill in the art.

Also encompassed by the invention are isolated RNAs transcribed from the nucleic acid sequences disclosed herein, as well as isolated nucleic acid molecules that hybridize to the nucleic acid sequences disclosed herein under stringent or moderately stringent hybridization conditions. The phrase “stringent hybridization conditions” refers to conditions under which a probe will hybridize to its target subsequence, typically in a complex mixture of nucleic acid, but to no other sequences. Stringent conditions are sequence dependent and will be different in different circumstances. Longer sequences hybridize specifically at higher temperatures. An extensive guide to the hybridization of nucleic acids is found in Tijssen, Techniques in Biochemistry and Molecular Biology—Hybridisation with Nucleic Probes, “Overview of principles of hybridization and the strategy of nucleic acid assays” (1993).

Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5× SSC, and 1% SDS, incubating at 42° C., or, 5xSSC, 1% SDS, incubating at 65° C., with wash in 0.2× SSC, and 0.1% SDS at 65° C. Such hybridizations and wash steps can be carried out for, e.g., 1, 2, 5, 10, 15, 30, 60; or more minutes.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1× SSC at 45° C. Such hybridizations and wash steps can be carried out for, e.g., 1, 2, 5, 10, 15, 30, 60, or more minutes. A positive hybridization is at least twice background. Those of ordinary skill will readily recognize that alternative hybridization and wash conditions can be utilized to provide conditions of similar stringency.

In another aspect of the invention, isolated fragments of the nucleic acid molecules of the invention are provided that are at least about 20 to 30 nucleotide bases in length. Such fragments may serve as primers for amplification or probes for the detection of orthologs of TSTP genes in other mammalian species. For such detection experiments, the considerations in choosing appropriate fragment length and relative quantities of G and C nucleotides (triple bond pairs) and A and T nucleotides (double bond pairs) and the corresponding hybridization conditions for achieving selective or specific hybridization to is well within the purview of the ordinarily skilled artisan. “Selective or specific hybridization” refers to the binding, duplexing, or hybridizing of a molecule only to a particular nucleotide sequence under stringent hybridization conditions when that sequence is present in a complex mixture (e.g., total cellular or library DNA or RNA).

As used herein, the terms “amplifying” and “amplification” refer to the use of any suitable amplification methodology for generating or detecting recombinant or naturally expressed nucleic acid, as described in detail, below. For example, the invention provides methods and reagents (e.g., specific degenerate oligonucleotide primer pairs) for amplifying (e.g., by polymerase chain reaction, PCR) naturally expressed (e.g., genomic or mRNA) or recombinant (e.g., cDNA) nucleic acids of the invention in vivo or in vitro.

The term “nucleic acid” or “nucleic acid sequence” refers to a deoxy-ribonucleotide or ribonucleotide oligonucleotide in either single or double-stranded form. The term encompasses nucleic acids, i.e., oligonucleotides, containing known analogs of natural nucleotides. The term also encompasses nucleic-acid-like structures with synthetic backbones (see e.g., F. Eckstein ed., Oligonucleotides and Analogues, a Practical Approach, Oxford Univ. Press (1991); Baserga et al eds., Antisense Strategies, Annals of the N.Y. Academy of Sciences, Vol. 600, (NYAS 1992); Milligan, J. Med. Chem., 36:1923-37(1993); Antisense Research and Applications, CRC Press, (1993); WO 97/03211; WO 96/39154; Mata, Toxicol. Appl. Pharmacol., 144:189-97 (1997); Strauss-Soukup, Biochemistry, 36:8692-98 (1997); Samstag, Antisense Nucleic Acid Drug Dev., 6:153-56 (1996)).

Unless otherwise indicated, a particular nucleic acid sequence also implicitly encompasses conservatively modified variants thereof (e.g. degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-08 (1985); Rossolini et al., Mol. Cell. Probes, 8:91-98 (1994)). The term nucleic acid is used interchangeably with gene, cDNA, mRNA, oligonucleotide, and polynucleotide.

In yet another aspect of the invention, chimeric or fused nucleic acid molecules are provided, wherein the chimeric or fused nucleic acid sequence comprises at least part of a TSTP coding sequence, at least part of a heterologous coding sequence, and transcription of the chimeric or fused nucleic acid sequence results in a single chimeric nucleic acid transcript. For instance, the heterologous coding sequence may be from a sequence encoding a different signal transduction polypeptide. Chimeras of the genes of the invention may be particularly useful for studying the function of various protein domains, i.e., where a chimera that functions in one mammalian species, but not in another might signal the presence of an sensory protein domain that interacts with another signal transduction protein which exhibits sequence variability between species. Chimeras of the invention might also be useful for identifying modulating compounds, and for isolating variants of sensory proteins of the invention showing either decreased or enhanced signal transduction activity.

Chimeric polypeptides containing heterologous coding sequences that facilitate expression of all or part of the TSTPs of the invention on the surface of cells can facilitate the creation of cellular libraries for the screening of compounds or other proteins which modulate signal transduction activity of TSTPs and/or taste perception. Alternatively, fusion proteins of the invention may be used to detect gene expression of TSTPs in various cells, or to analyze environmental effects on the transcription of the genes of the invention. In this regard, the heterologous coding sequence may be from a gene encoding green fluorescent protein.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

For analyzing the effects of various TSTP genes in different cellular or species backgrounds, the nucleic acid sequences disclosed herein may be operably linked to a heterologous promoter that is either regulatable or constitutive. A regulatable promoter is most valuable in this context, because it is inducible under specific environmental or developmental conditions. Such a promoter is also useful where expression of the TSTP is detrimental to the transfected host cell, and expression can be turned on only for a short while or at a certain stage for the purpose of measuring the effects of gene expression.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

Promoter-gene constructs may optionally be cloned into an expression vector before being transfected into a host cell. However, it is also possible to transfect free DNA such that it contains flanking DNA that allows integration into the genome. Any type of expression vector may be used to clone, propagate and express the nucleic acid molecules disclosed herein, including mammalian vectors, bacterial plasmids, bacterial phagemids, mammalian viruses and retroviruses, and bacteriophage vectors. Transfected host cells and transgenic animals expressing the nucleic acid sequences of the invention, or having such sequences deleted, are also encompassed in the present invention. Methods of transfecting cells and making transgenic animals are well within the skill of the art (see, e.g., Bijvoet, Hum. Mol. Genet., 7:53-62 (1998); Moreadith, J. Mol. Med., 75:208-16 (1997); Tojo, Cytotechnology, 19:161-65 (1995); Mudgett, Methods Mol. Biol. 48:167-84 (1995); Longo, Transgenic Res. 6:321-28 (1997); U.S. Pat. Nos. 5,616,491; 5,464,764; 5,631,153; 5,487,992; 5,627,059; 5,272,071; WO 91/09955; WO93/09222; WO 96/29411; WO 95/31560; WO 91/12650. “Knock out” transgenic animals, e.g., having a particular paralog member of a TSTP gene deleted in the germ line, may be useful for elucidating the function of TSTP in various cell types.

In another aspect of the invention, the nucleic acids and polypeptides disclosed herein may be coupled to a solid support for the purpose of identifying molecules, proteins, or compounds which bind to the nucleic acids and proteins described herein. Alternatively, the nucleic acids of the invention may be used to screen libraries, i.e., gene chip arrays, to identify paralog and ortholog members of the present invention. Transfected host cells, or individual polypeptides or nucleic acids may also be used for identifying binding molecules, proteins or compounds.

For instance, in one embodiment of the invention, a method of screening for compounds that activate TSTP related signal transduction is provided comprising: (i) contacting a host cell expressing a TSTP with a putative signal transduction modulating compound; and (ii) measuring the activity from generated from said TSTP expressed in said cell. The host cells used for such methods may also be transfected with genes encoding other taste-specific signal transduction molecules, such as genes for taste G Protein-Coupled Receptors and for G Proteins that interact with such receptors. Particularly preferred are promiscuous G proteins such as Gα15 or Gα16, or other Gα proteins that facilitate signal transduction from a wide range of G Protein-Coupled Receptors. Such proteins are described in copending application Ser. No. 243,770, which is herein incorporated by reference in its entirety.

Such screening may also be performed by methods comprising: (i) contacting a host cell expressing a TSTP gene with a known taste activating compound and a compound putatively involved in taste transduction modulation; (ii) contacting a similar host cell with a known taste activating compound alone; and (iii) comparing the activity from the TSTP expressed in the host cell of step (i) with the activity from host cell of step (ii) to identify modulators of taste signal transduction. The modulatory compounds identified by the methods of the present invention can include activators, inhibitors, stimulators, enhancers, agonists and antagonists, all of which are also the subject of the invention.

Compounds tested as modulators of TSTP signaling can be any small chemical compound, or a biological entity, such as a protein, sugar, nucleic acid or lipid. Alternatively, modulators can be genetically altered versions of TSTPs. Typically, test compounds will be small chemical molecules and peptides. Essentially any chemical compound can be used as a potential modulator or ligand in the assays of the invention. The assays are designed to screen large chemical libraries by automating the assay steps and providing compounds from any convenient source to assay, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), and the like.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used in consumer products.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res., 37:487-93 (1991) and Houghton et al, Nature, 354:84-88 (1991)). Other chemistries for generating chemical diversity libraries can also be used. Such chemistries include, but are not limited to: peptoids (e.g., WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (Hobbs et al., PNAS, 90:6909-13 (1993)), vinylogous polypeptides (Hagihara et al., J. Amer. Chem. Soc., 114:6568 (1992)), nonpeptidal peptidomimetics with glucose scaffolding (Hirschrnann et al, J. Amer. Chem. Soc., 114:9217-18 (1992)), analogous organic syntheses of small compound libraries (Chen et al., J. Amer. Chem. Soc., 116:2661 (1994)), oligocarbamates (Cho et al., Science, 261:1303 (1993)), peptidyl phosphonates (Campbell et al., J. Org. Chem., 59:658 (1994)), nucleic acid libraries (Ausubel, Berger, and Sambrook, all supra), peptide nucleic acid libraries (U.S. Pat. No. 5,539,083), antibody libraries (Vaughn et al., Nature Biotechnology, 14(3):309-14 (1996) and PCT/US96/10287), carbohydrate libraries (Liang et al., Science, 274:1520-22 (1996) and U.S. Pat. No. 5,593,853), small organic molecule libraries (benzodiazepines, Baum, C&EN, January 18, page 33 (1993); thiazolidinones and metathiazanones, U.S. Pat. No. 5,549,974; pynrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS (Advanced Chem Tech, Louisville Ky.), Symphony (Rainin, Woburn, Mass.), 433A (Applied Biosystems, Foster City, Calif.), 9050 Plus (Millipore, Bedford, Mass.)). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences; Columbia, Md.; etc.).

In one aspect of the invention, the modulators identified using the TSTPs disclosed herein can be used in any food product, confectionery, pharmaceutical composition, or ingredient thereof to thereby modulate the taste of the product, composition, or ingredient in a desired manner. For instance, such modulators can be used to disrupt or enhance a signaling cascade involved in taste signaling to thereby block or enhance taste perception.

Also included in the present invention are isolated polypeptides comprising an amino acid sequence selected from the group consisting of: (i) an amino acid sequence encoded by a nucleic acid sequence having at least 75%, 80%, 85%, 90% 95%, 96%, 97%, 98%, or 99% identify to a sequence selected from SEQ ID NOS: 1, 2, 3, 5, 7, 9, 11, 12, 15, 16, 17, 18, 19, 20, 22, and 24; (ii) a TSTP encoded by a DNA sequence having at least about 75% identity to a sequence selected from the group consisting of SEQ ID NOS: 4, 6, 8, 10, 12, 14, 21, and 23; (iii) a variant of a TSTP encoded by a nucleotide sequence selected from the group consisting of SEQ ID NOS: 4, 6, 8, 10, 12, 14, 21, and 23, wherein said variant contains at least one conservative substitution relative to a TSTP encoded by said nucleotide sequence; and (vi) a variant of a TSTP comprising an amino acid sequence selected from the group consisting of SEQ ID NOS: 1, 2, 3, 5, 7, 9, 11, 12, 15, 16, 17, 18, 19, 20, 22, and 24, wherein said variant contains at least one conservative substitution.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. “Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein.

Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (I); 5) Isoleucine (I), Leucine (L), Methionine (M), Valine (V); and 6) Phenylalanine (F), Tyrosine (Y), Tryptophan (W); (see also, e.g., Creighton, Proteins, W.H. Freeman and Company (1984); Schultz and Schimer, Principles of Protein Structure, Springer-Verlag (1979)). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

The terms “mimetic” and “peptidomimetic” refer to a synthetic chemical compound that has substantially the same structural and/or functional characteristics of the polypeptides. The mimetic can be either entirely composed of synthetic, non-natural analogs of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity.

A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄), thiazole, retroamide, thioamide, or ester (see, e.g., Spatola, Chemistry and Biochemistry of Amino Acids, Peptides and Proteins, 7:267-357 (1983)). A polypeptide can also be characterized as a mimetic by containing all or some non-natural residues in place of naturally occurring amino acid residues; non-natural residues are well described in the scientific and patent literature.

Also included in the invention are polypeptide fragments of the TSTPs described herein, wherein said fragments comprises at least about 5 to 7 amino acids. Particularly preferred are fragments containing a functional domain of a TSTP, wherein the domain plays a role in the signal transduction of taste perception, or interacts with a compound involved in taste activation or modulation or another signal transduction protein. Chimeric and fusion proteins are also included. Such fragments and full length polypeptides are useful in methods of screening one or more compounds for the presence of a compound that activates or modulates signal transduction, wherein said one or more compounds are contacted with a TSTP fragment or full-length polypeptide. In particular, such methods are used to identify compounds that interact with or modulate the activity of at least one TSTP in taste receptor cells. Polypeptide arrays comprising TSTPs or polypeptide segments of the invention may also be employed for screening assays. Polypeptides in arrays may be linked covalently or noncovalently to a solid phase support, or may be expressed in vivo in phage display libraries, or such that they are displayed on the surface of cells.

Isolated antibodies that bind with specificity to TSTPs of the invention may be isolated using known methodology, and are also a subject of the invention. “Antibody” refers to a potypeptide comprising a framework region from an immunoglobulin gene or fragments thereof that specifically binds and recognizes an antigen. Any antibody having specificity for a TSTP is included, where specificity is denoted by the ability to bind to a TSTP or fragments thereof, and not to any other signal transduction or other proteins. “Antibodies” is inclusive of whole antibodies, fragments, e.g., Fv, Fab′ or (Fab)′₂ fragments, chimeric antibodies, humanized antibodies, etc. A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

Methods of producing polyclonal and monoclonal antibodies that react specifically with a TSTP or fragment thereof are known to those of skill in the art (see, e.g., Coligan, Current Protocols in Immunology (1991); ., Harlow & Lane, Antibodies, A Laboratory Manual, (1988); Goding, Monoclonal Antibodies: Principles and Practice (2d ed. 1986); and Kohler & Milstein, Nature, 256:495-97 (1975)). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science, 246:1275-81 (1989); Ward et al., Nature, 341:544-46 (1989)).

A number of TSTP-comprising immunogens may be used to produce antibodies specifically reactive with a TSTP. For example, a recombinant TSTP, or an antigenic fragment thereof, can be isolated as described herein. Suitable antigenic regions include, e.g., the conserved motifs that may be used to identify members of the various TSTP families. Recombinant proteins can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring proteins may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. For instance, an inbred strain of mice (e.g., BALB/C mice) or rabbits may be immunized with the protein using a standard adjuvant, such as Freund's adjuvant, and a standard immunization protocol. The animal's immune response to the immunogen preparation is then monitored by taking test bleeds and determining the titer of reactivity to the TSTP. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Monoclonal antibodies may be obtained by various techniques familiar to those skilled in the art. For example, spleen cells from an animal immunized with a desired antigen may be immortalized, commonly by fusion with a myeloma cell (see Kohler & Milstein, Eur. J. Immunol., 6:511-19 (1976)). Alternative methods of immortalization include transformation with Epstein Barr Virus, oncogenes, or retroviruses, or other methods well known in the art. Colonies arising from single immortalized cells are then screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science, 246:1275-81 (1989).

Monoclonal antibodies and polyclonal sera may be collected and titered against the immunogen protein in an immunoassay, for example, a solid phase immunoassay with the immunogen immobilized on a solid support. Typically, polyclonal antisera with a titer of 104 or greater are selected and tested for their cross reactivity against a non-TSTP, another TSTP family member, or other related proteins from other organisms, using a competitive binding immunoassay. Specific polyclonal antisera and monoclonal antibodies will usually bind with a Kd of at least about 0.1 mM, more usually at least about 1 pM, optionally at least about 0.1 pM or better, and optionally 0.01 pM or better.

Once TSTP specific antibodies are available, individual TSTPs or fragments thereof can be detected by a variety of immunoassay methods. For a review of immunological and immunoassay procedures, see Basic and Clinical Immunology (see Stites & Terr eds., 7th ed. (1991)). Moreover, the immunoassays of the present invention can be performed in any of several configurations, which are reviewed extensively in Enzyme Immunoassay (see Maggio ed. (1980); and Harlow & Lane, supra).

An “anti-TSTP” antibody is an antibody or antibody fragment that specifically binds a polypeptide encoded by a TSTP gene, cDNA, or a subsequence thereof. The phrase “specifically (or selectively) binds” to an antibody or, “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular TSTP at least two times the background and do not substantially bind in a significant amount to other proteins present in the sample. Specific binding to an antibody under such conditions may require an antibody that is selected for its specificity for a particular protein.

For example, polyclonal antibodies raised to a TSTP from a specific species such as rat, mouse, or human can be selected to obtain only those polyclonal antibodies that are specifically immunoreactive with that TSTP or an immunogenic portion thereof, and not with other proteins, except for orthologs or polymorphic variants and alleles of the TSTP of interest. This selection may be achieved by subtracting out antibodies that cross-react with TSTPs from other species or other family members. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, supra, for a description of immunoassay formats and conditions that can be used to determine specific immunoreactivity). Typically a specific or selective reaction will be at least twice background signal or noise and more typically more than 10 to 100 times background.

TSTPs, fragments, or variants thereof can be detected and/or quantified using any of a number of well recognized immunological binding assays (see, e.g., U.S. Patents 4,366,241; 4,376,110; 4,517,288; and 4,837,168). For a review of the general immunoassays, see also Asai, ed., Methods in Cell Biology: Antibodies in Cell Biology, 37 (1993); Stites & Terr, eds., Basic and Clinical Immunology (7th ed. 1991). Immunological binding assays (or immunoassays) typically use an antibody that specifically binds to a protein or antigen of choice (in this case a TSTP or an antigenic subsequence thereof). The antibody (e.g., anti-TSTP) may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled TSTP or a labeled anti-TSTP antibody. Alternatively, the labeling agent may be a third moiety, such a secondary antibody, that specifically binds to the antibody/TSTP complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol., 111:1401-06 (1973); Akerstrom et al., J. Immunol, 135:2589-42 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Immunoassays for detecting a TSTP in a sample may be either competitive or noncompetitive. Noncompetitive immunoassays are assays in which the amount of antigen is directly measured. In one preferred “sandwich” assay, for example, the anti-TSTP antibodies can be bound directly to a solid substrate on which they are immobilized. These immobilized antibodies then capture the TSTP or fragment thereof present in the test sample. The TSTP thus immobilized is then bound by a labeling agent, such as a second TSTP antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

In competitive assays, the amount of TSTP present in the sample is measured indirectly by measuring the amount of a known, added (exogenous) TSTP competed away from an anti-TSTP antibody by the unknown TSTP present in a sample. In one competitive assay, a known amount of TSTP is added to a sample, and the sample is then contacted with an antibody that specifically binds to the TSTP. The amount of exogenous TSTP bound to the antibody is inversely proportional to the concentration of TSTP present in the sample. In a particularly preferred embodiment, the antibody is immobilized on a solid substrate. The amount of TSTP protein bound to the antibody may be determined either by measuring the amount of TSTP present in a TSTP/antibody complex, or alternatively by measuring the amount of remaining uncomplexed protein. The amount of TSTP may optionally be detected by providing a labeled TSTP molecule.

A hapten inhibition assay is another preferred competitive assay. In this assay the known TSTP is immobilized on a solid substrate. A known amount of anti-TSTP antibody is added to the sample, and the sample is then contacted with the immobilized TSTP. The amount of antibody bound to the known immobilized protein is inversely proportional to the amount of protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Immunoassays in the competitive binding format can also be used for cross-reactivity determinations. For example, a protein at least partially encoded by the nucleic acid sequences disclosed herein can be immobilized to a solid support.

Proteins (e.g., TSTPs and homologs) are added to the assay that compete for binding of the antisera to the immobilized antigen. The ability of the added proteins to compete for binding of the antisera to the immobilized protein is compared to the ability of the TSTP to compete with itself. The percent cross-reactivity for the above proteins is calculated, using standard calculations. Those antisera with less than 10% cross-reactivity with each of the added proteins listed above are selected and pooled. The cross-reacting antibodies are optionally removed from the pooled antisera by immunoabsorption with the added considered proteins, e.g., distantly related homologs. In addition, peptides comprising amino acid sequences representing conserved motifs that may be used to identify TSTPs can be used in cross-reactivity determinations.

The immunoabsorbed and pooled antisera are then used in a competitive binding immunoassay as described above to compare a second protein, thought to be perhaps an allele or polymorphic variant of a TSTP, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein encoded by nucleic acid sequences disclosed herein required to inhibit 50% of binding, then the second protein is said to specifically bind to the polyclonal antibodies generated to a TSTP immunogen.

Polyclonal antibodies that specifically bind to a particular TSTP can be made by subtracting out cross-reactive antibodies using other TSTPs. Species-specific polyclonal antibodies can be made in a similar way. For example, antibodies specific to human TSTP proteins can be made by, subtracting out antibodies that are cross-reactive with orthologous sequences, e.g., rat or mouse TSTPs.

Western blot (immunoblot) analysis may also be used to detect and quantify the presence of a TSTP in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the TSTPs. The anti-TSTP antibodies specifically bind to the TSTPs on the solid support. These antibodies may be directly labeled or alternatively may be subsequently detected using labeled antibodies (e.g., labeled sheep anti-mouse antibodies) that specifically bind to the anti-TSTP antibodies.

The invention is further illustrated by the following non-limiting Examples.

### EXAMPLES

In the protein sequences presented herein, the codes X or Xaa refers to any of the twenty common amino acid residues. In the DNA sequences presented herein, the codes N or n refers to any of the of the four common nucleotide bases, A, T, C, or G.

The following examples provides the nucleotide and conceptually-translated protein sequences for the disclosed genes (including the sequence of the C. elegans gene product re-derived using an alternative splice junction), which are identified in this application and the accompanying sequence listing as SEQ ID NOS. 1-24.

EXAMPLE 1—rat 165-015 TSTPs
r165-015A (rat TCP #3) conceptual translation (SEQ ID NO 1)
MDRFRMLFQNFQSSSESVTNGICLLLAAVTVKMYSSLDFNCPCLERYNALYGLGLLLTPPLAL
FLCGLLVNRQSVLMVEEWRRPAGHRRKDLGIIRYMCSSVLQRALAAPLVWILLALLDGKCLV
CAFSNSVDPEKFLDFANMTPSQVQLFLAKVPCKEDELVKTNPARKAYSRYLRCLSQAIGWSIT
LLVIVVAFLARCLRPCFNQTVFLQRRYWSNYMDLEQKLFDETCCEHARDFAHRCVLHFFASM
QSELRALGLHRDPAGEILESQEPPEPPEEPGSESGKAHLRAISSREQVNHLLSTWYSSKPPLDLA
ASPRLWEPGLNHRAPTAAPGTKLGHQLDV (SEQ ID NO 1)
r165-015B conceptual translation (SEQ ID NO 2)
r165-015B was assembled from EST A1146249
AAALAPLTWVAVALLGGAFYECAATGSAAFAQRLCLGRNRSCAAELPLVPCNQAKASDVQD
LLKDLKAQSQVLGWILIAVVIIILLIFTSVTRCLSPVSFLQLKFWKIYLEQEQQILKATEHATE
LAKENIKCFFEGSHPKEYNTPSMKEWQQISSLYTF (SEQ ID NO 2)
r165-015C conceptual translation (SEQ ID NO 3)
r165-015C was assembled from EST AA998169
RSTLVCAQVLGWVLIAAVIFLLLVFKCVSRCFSPVSYLQLKJWEIYLEKEKQTLQSQAAEHATQ
LARENIRSFFECSIQPKECNTPSRKDWQQISALYTFNSKNQFYSMLHKYVSRKVSSSLHSVEGD
VVVPVLGFVDDAAMANTHGV (SEQ ID NO 3)
EXAMPLE 2—human 165-015 TSTPs
h165-015A Genomic DNA (SEQ ID NO 4)
h165-015A was identified from chr 10 HTGS BAC, accession number AL139339. Some inuonic
sequence intervals are denoted as runs of N and the predicted coding sequence is denoted in boldface.
GCTCGTCCCCAGCACAGCAGACACCAGGAAGGTGGCCAGAGCCTCACTGAGCCGAACCG
ACGGCCGCCCACCCACCCAGGCTGGAGCCATGGATAAATTCCGCATGCTCTTCCAGCAC
TTCCAGTCAAGCTCGGAGTCGGTGATGAATGGCATCTGCCTGCTGCTGGCTGCGGTC
ACCGTCAAGCTGTACTCCTCCTTTGACTTCAACTGTCCCTGCCTGGTGCACTACAATG
CACTCTACGGCCTGGGCCTGCTGCTGACGCCCCCGCTCGCCCTGTTTCTCTGCGGCC
TCCTCGCCAACCGGCAGTCTGTGGTGATGGTCGAGGAGTGGCGCCGGCCCGCAGGG
CACCGGAGGAAGGACCCAGGCATCATCAGGTGCGGTCCCACACCACTCAGTGACCCAT
GAGCTTTGCCAGGGGCTCCCCAGCCACCCCACAGGCACTGTCAAGNNNNNNNNNNNNNN
NNNNNNNNNNNNNNNNNNNNNNNNNNNNNNNNNNNNNTCTGAGTAACCCAGCCTGGGAC
TTCCCAGCAGTCCATCCTTGCACCTCCCCTGCTGCAGCTGACTTCCAGCTGCCCCTTGACCC
CCCTCCTTCCTCTGCTGCCAGGTACATGTGCTCCTCTGTGCTGCAGAGGGCGCTGGCC
GCCCCCCTGGTCTGGATCCTGCTGGCCCTCCTTGACGGGAAGTGCTTCGTGTGTGCC
TTCAGCAGCTCTGTGGACCCTGAGAAGTTTCTGGACTTTGCCAACATGACCCCCAGC
CAGGTACAGCTCTTCCTGGCCAAGGTTCCCTGCAAGGAGGATGAGCTGGTCAGGGA
TAGCCCTGCTCGGAAGGCAGTGTCTCGCTACCTGCGGTGCCTGTCACAGGTAACTGG
GGTGATCCTGCCCCGGCCCTTGCACCCTCACAAATCCCCCGCATTGGTTCTGGAGTGGGGT
NAGGGGTGGTGCAAGAAGGGNNNNNNNNNNNNNNNNNNNNNNNNNNNNNNNNNNNNNN
NNNNNNNNNNNNNTATTCATAAATACATATGTATATAGGATTAGGCCCCACTCACATCTA
ACCTCGGGCCTCCTTACTTTGCAGGCCATCGGCTGGAGCGTCACCCTGCTGCTGATCAT
CGCGGCCTTCCTGGCCCGCTGCCTGAGGCCCTGCTTCGACCAGACAGTCTTCCTGCA
GCGCAGATACTGGAGCAACTACGTGGACCTGGAGCAGAAGCTCTTCGACGAGACCT
GCTGTGAGCATGCGCGGGACTTCGCGCACCGCTGCGTGCTGCACTTCTTTGCCAGCA
TGCGGAGTGAGCTGCAGGCGCGCGGGGCTGCGCCGGGGCAATGCAGGCAGGAGACT
CGAGCTCCCCGCAGTGCCTGAGCCCCCAGAAGGCCTGGATAGTGGAAGTGGGAAGG
CCCACCTGCGCGCAATCTCCAGCCGGGAGCAGGTGGACCGCCTCCTAAGCACGTGG
TACTCCAGCAAGCCGCCGCTGGACCTGGCTGCATCCCCCGGGCTCTGCGGGGGTGG
CCTTAGCCACCGCGCCCCTACCTTGGCACTGGGCACGAGGCTGTCACACACACCGA
CGTGTAGGGTCCTGGCCAGGCTTGAAGCGGCAGTGTTCGCAGGTGAAATGCCGCGCTGAC
AAAGTTCTGGAGTCIITCCAGGCCGTGGGGACCCCACGGCAGGCACCCTAAGTCTTGTTA
GCCTCCTTTTTAAAGTAGCCCAATCTCTGCCTAGTTTCTGGGTGTGGCCTCCAGCGCGCTTC
ACAAACTTTAATGTGGACTCGGTTCACCGAGGGCCTTGTTAAATACAGGTTCAGACAGTGT
A (SEQ ID NO 4)
h165-015A conceptual translation (SEQ ID NO 5)
MDKYRMLFQHFQSSSESVMNGICLLLAAVTVKLYSSFDFNCPCLVHYNALYGLGLLLTPPLAL
FLCGLLANRQSVVMVEEWRRPAGHRRPGIIRYMCSSVLQRALAAPLVWTLLALLDGKCFV
CAFSSSVDPEKiPLDFANMTPSQVQLFLAKVPCKEDELVRDSPAPVSRYLRCLSQAIGWSVT
LLLIIAAFLARCLRPCEDQTVELQRRYWSNYVDLEQKLFDETCCEHAPJJFAHRCVLHFFASMR
SELQARGLRRGNAGRRLELPAVPEPPEGLDSGSGKAHLRAISSREQVDRLLSTWYSSPPLDLA
ASPGLCGGGLSHRAPTLALGTRLSQHTDV (SEQ ID NO: 5)
h165-015B genomic DNA sequence (SEQ ID NO 6)
h165-015B was identified from dir 10 HTGS BAG, accession number AL139339. Some intronic
sequence intervals are denoted as runs of N and predicted coding sequence is denoted in boldface
CCAGCAACCATCAATCCCGTCTCCTCCTGCCTCCTCTGCTGCAATCCACCCCGCCACGACT
ATCGCCATGGCAGCCCTGATCGCAGAGAACTTCCGCTTCCTGTCACTTTTCTTCAAGA
GCAAGGATGTGATGATTTTCAACGGCCTGGTGGCACTGGGCACGGTGGGCAGCCAG
GAGCTGTTCTCTGTGGTGGCCTTCCACTGCCCCTGCTCGCCGGCCCGGAACTACCTG
TACGGGCTGGCGGCCATCGGCGTGCCCGCCCTGGTGCTCTTCATCATTGGCATCATC
CTCAACAACCACACCTGGAACCTCGTGGCCGAGTGCCAGCACCGGAGGACCAAGAA
CTGCTCCGCCGCCCCCACCTTCCTCCTTCTAAGCTCCATCCTGGGACGTGCGGCTGT
GGCCCCTGTCACCTGGTCTGTCATCTCCCTGCTGCGTGGTGAGGCTTATGTCTGTGC
TCTCAGTGAGTTCGTGGACCCTTCCTCACTCACGGCCAGGGAAGAGCACTTCCCATC
AGCCCACGCCACTGAAATCCTGGCCAGGTTCCCCTGCAAGGAGAACCCTGACAACCT
GTCAGACTTCCGGGAGGAGGTCAGCCGCAGGCTCAGGTATGAGTCCCAGGTAAGGA
GCTGTGCAAAGGGAAGCTCCTCTTCCCTAGTGGTGGCTGGTGAGAGGTCCGGNNNNNNNN
NNNNNNNNNNNNNNNNNNNNNNNNNTTCCATTCGCTCTOGGGGGTAGGGCTTAGGCAGTT
CCTCGCCCCTCCTGAACTGTGCCCATTCTCTGGCCAGCTCTTTGGATGGCTGCTCATCGG
CGTGGTGGCCATCCTGGTGTTCCTGACCAAGTGCCTCAAGCATTACTGCTCACCACT
CAGCTACCGCCAGGAGGCCTACTGGGCGCAGTACCGCGCCAATGAGGACCAGCTGT
TCCAGCGCACGGCCGAGGTGCACTCTCGGGTGCTCGCTGCCAACAATGTGCGCCGC
TTCTTTGGCTTTGTGGCGCTCAACAAGGATGATGAGGAACTGATTGCCAACTTCCCA
GTGGAAGGCACGCAGCCACGGCCACAGTGGAATGCCATCACCGGCGTCTACTTGTA
CCGTGAGAACCAGGGCCTCCCACTCTACAGCCGCCTGCACAAGTGGGCCCAGGGTC
TGGCAGGCAACGGCGCGGCCCCTGACAACGTGGAGATGGCCCTGCTCCCCTCCTAA
GGAGGTGCTTCCCATGCTCTTTGTAAATGGCACTACTTGGTCCCAAACTGAACCCCACTGC
TTGCTCACATCCATATCAGAAGGGGATTTTTAAAAAACTGTTATCTCTTGGCCAGGGGAA
AGGACCACAAGGCAATCTGGGGTGTGGACAGACCCAGTAGACAATGGAAGCCCCAGCCA
GCAGGGCCAGGTGACAGTGAAGCTCACCAGTGGGCTCCTTTATGGTACTCTATGCAGTTA
ACATGTATCTAGCTGCATAGGGACACCCAGCGCAGCAGTGCACCACTGGGAAGTGGCCTC
(SEQ ID NO 6)
h165-015B (full-length of partial human 1 TCP #3) conceptual
translation (SEQ ID NO 7)
MAALIAENFRFLSLFFKSKDVMIFNGLVALGTVGSQELFSVVAFHCPCSPARNYLYGLAAIGVP
ALVLFIIGIJLNHTWNLVAECQHRRTKNCSAAPTFLLLSSILGRAAVAPVTWSVISLLRGEAYV
CALSEFVDPSSLTAREEHFPSAHATEILARFPCKENPDNLSDFREEVSRRLRYESQLFGWLLIGV
VAILVFLTKCLKHYCSPLSYRQEAYWAQYRANEDQLFQRTAEVHSRVLAANNVRRFFGEVAL
NKDDEELIANFPVEGTQPRPQWNAITGVYLYRENQGLPLYSRLHKWAQGLAGNGAAPDNWB
MALLPS (SEQ ID NO 7)
h165-015C (human 2 TCP #3) genomic DNA (SEQ ID 8)
h165-015C was identified from cbr 6 PAC, accession number Z84488. The full length genomic
sequence ends at poly(A) site based on cDNA clone AF086 130. Some intronic sequence intervals are
denoted as runs of N and the predicted coding sequence is denoted in boldface.
TGCCTCTGCCTTTGGAATGGGGTGGGTGCCAGAGAAGTCAAGGTCCTGGGGGTGTTTGCTT
GCCTGGTTCTGTTAACCAGAGGTGGTGTGGCTGAAATGAGAGTGAAACTTTAGGAAGG
TCTGAGAAGCCCTCTTCCCTTTAAAAAAAAAAAAAAAGGCTGCTTCTCGCAGAGTGGA
AAGCCCCGGTCCCCATCCCACCAAACCATTTGACAAGCAGGACAACGAAGAGGCAGAA
GGATCTGGGCCTGTGCGCGACGCCCCGGGGGACGAGGCTCATGGAGAAGTTTCGGGCG
GTGCTGGACCTGCACGTCAAGCACCACAGCGCCTTGGGCTACGGCCTGGTGACCCT
GCTGACGGCGGGCGGGGAGCGCATCTTCTCCGCCGTGGCATTCCAGTGCCCGTGCA
GCGCCGCCTGGAACCTGCCCTACGGCCTGGTCTTCTTGCTGGTGCCGGCGCTCGCG
CTCTTCCTCCTGGGCTACGTGCTGAGCGCACGCACGTGGCGCCTGCTCACCGGATGC
TGCTCCAGCGCCCGCGCGAGTTGCGGATCGGCGCTGCGCGGCTCCCTGGTGTGCAC
GCAAATCAGCGCGGCCGCCGCGCTCGCGCCCCTCACCTGGGTGGCCGTGGCGCTGC
TCGGGGGCGCCTTTTACGAGTGCGCGGCCACCGGGAGCGCGGCCTTCGCGCAGCGC
CTGTGCCTCGGCCGCAACCGCAGCTGCGCCGCGGAGCTGCCGCTGGTGCCGTGCAA
CCAGGCCAAGGCGTCGGACGTGCAGGACCTCCTGAAGGATCTGAAGGCTCAGTCGC
AGGTCTGCCGCTGGCGCTGGGGGCGTTTGGGAGGAGCCGAGAGGCCGAGCTTTCTCAGGG
CCGCTGGGGTGAGGGAAAAATCGGTGACTTTTCTCCAGATATACAGTACCCTAAGAAAAT
CTAGAATGGCTCCTTGCATCTAATTTGCCGTCAAGAGAATATCTGAATAAAACGAATGAA
AAGGAGAAAAACGCATTCCCGTAGTATCTGGCACTGTACATGAATCGTGGAAAGTGGGAG
TGAGAGTGGGCACGCCTACTCAGTGCCAGATACTGCGCTAGGCCCTTGACCTCCTGCATTA
TTTTTACGTCTCACAACAGCTCTGTTGGGTGCAATTGCGGTTTTGCCATTACAAGTAAT
AGTTTCGTATCTCCCCTATATAGTGACTTCACTGAGTCTAAGTAAAGTTGTTTGCCCAGCC
AATAGAAAGCAGAATTGTTAGAATTAAAGGGGTCTATAGAATAAAATGGGTTTGTCTGGC
TCTAAAGCCACCACTGGACTTAGAAAGCTCAGAGGTTCTTTAAAATGAACACTTCTTTCCC
AGGATTTGCAGAAATTCCACATTTTGAGTTCTAAGGGAATGGTATGGGCTCCTCCCTGGGC
AGAATCATAGTGCATAACTTGGACTACAGTTGACTTTCCAAACGAGAAGTTGTACTGGGG
GGCTAGGGAGGTTACTGCGACACCTTACTCTTAAGGAATTAAGACCTAAAACTGTTGCTTG
TTCATCATCATAACTGGCGAGTAGTTGAAACTTTATCTGAGGATTTTTAAATTTCTTGTAA
AAAACCAAGTAACTAAATTACTATTTTGTTGTGTTTTCTTAAGGTGTTGGGCTGGATCTT
GATAGCAGTTGTTATCATCATTCTTCTGATTTTTACATCTGTCACCCGATGCCTATCT
CCAGTTAGTTTTCTGCAGCTGAAATTCTGGAAAATCTATTTGGAACAGGAGCAGCAG
ATCCTTAAAGTAAGCCACAGAGCATGCAACTGAATTGGCAAAAGAGAATATTAAA
TGTTTCTTTGAGGGCTCGCATCCAAAAGAATATAACACTCCAAGCATGAAAGAGTGG
CAGCAAATTTCATCACTGTATACTTTCAATCCGAAGGGCCAGTACTACAGCATGTTG
CACAAATATGTCAACAGAAAAGAGAAGACTCACAGTATCAGGTCTACTGAAGGAGAT
ACGGTGATTCCTGTTCTTGGCTTTGTAGATTCATCTGGTATAAACAGCACTCCTGAGT
TATGACCTTTTGAATGAGTAGKAAAAAATTGTTTTGAATTATTGCTTTATTAAAAAATA
AACATTGGT (SEQ ID NO 8)
h165-015C (human 2 TCP #3) conceptual translation (SEQ ID NO 9)
MEVLDLHVKHHSALGYGLVTLLTAGGERIFSAVAFQCPCSAAWNLPYGLVFLLVPALA
LFLLGYVLSARTWRLLTGCCSSARASCGSALRGSLVCTQISAAAALAPLTWVAVALLGGAFY
ECAATGSAAFAQRLCLGR4RSCAAELPLVPCNQAKASDVQDLLKDLKAQSQVLGWILIAVVII
ILLIFTSVTRCLSPVSFLWLKFWKIYLEWEWWILKSKATEHATELAKENIKCFFEGSHPKEYNTPS
MKEWQQJSSLYTFNPKGQYYSMLHKYVNPXEKTHSIRSTEGDTVIPVLGFVDSSGINSTPEL
(SEQ ID NO 9)
h165-015D genomic DNA (SEQ ID NO 10)
h165-015D was identified from 6q22.1-22.33 BAC, accession number AL121953. Some intronic
sequence intervals are denoted as runs of N and predicted coding sequence is denoted in boldface.
AGGAATGACATTGTTTCCTTATCTGGCTCAATTCAGTCTGAGAAGGACTGTTGTTTCTGAT
GAAGAAAGAGCTCCACCCTCGGCCACTGCCACAGCTGCTCTGCCAATAACAAAAGGCACAG
CATTYYCCCTCTGTGCATCTCCAACATGGATGCTTTTCAGGGCATTTTAAAATTCTTCCT
TAATCAGAAAACTGTTATTGGCTACAGCTTCATGGCTCTGCTGACCGTGGGAAGTGA
GCGTCTCTTTTCTGTTGTGGCTTTTAAGTGCCCCTGCAGCACTGAGAATATGACCTAT
GGGCTGGTTTTCCTCTTTGCTCCTGCCTGGGTGTTACTGATCCTGGGATTCTTTCTGA
ACAATAGGTCGTGGAGACTCTTCACAGGCTGCTGTGTGAATCCCAGGAAAATCTTTC
CCAGAGGCCACAGCTGCCGTTTCTTCTACGTCCTCGGCCAGATCACTCTGAGCTCAT
TGGTGGCTCCAGTGATGTGGCTTTCTGTGGCTCTGCTCAATGGAACTTTCTATGAAT
GTGCCATGAGCGGGACGAGAAGTTCAGGACTCCTGGAACTGATTTGCAAGGGTAAG
CCCAAAGAGTGCTGGGAAGAACTTCACAAAGTATCTTGTGGCAAAACTAGCATGCTA
CCTACCGTCAATGAAGAACTGAAACTCTCCCTTCAGGCCCAGTCTCAGGTAAGAAAA
GACAAACTCGCCTTTTTCTCTCAGCATGAGCTCGAAGTATCTCTCTGTGCTCCTTTCAGCC
AGGGCTGTCTGTGTCCTGTCAGAATATTTTGAAACTAAATGGANNNNNNNNNNNNNNNNN
NNNNNNNNNNNNNNNNNNNNNNNNNNNNNNNNNGACTGTGGTAATAATTTAGCTATACA
CTTCTCAATAATACCCCAGCTCCCTAAATGGCTTTGCTTCACGTGTGTACTCAATATTTCT
TTCTTCTTAAGATTCTAGGATGGTGCCTGATTTGTTCAGCGTCTTTCTTCTCTCTGCTC
ACCACATGTTATGCTCGCTGCCGATCTAAAAGTTAGCTACCTTCAGCTGAGTTTTTGGA
AGACATATGCACAAAAGGAGAAGGAGCAGTTGGAAAATACATTTCTGGACTATGCCA
ACAAGCTGAGCGAGAGGAACCTGAAATGTTTTTTTGAAAACAAGAGGCCAGATCCTT
TTCCCATGCCTACGTTTGCTGCCTGGGAGGCTGCTTCAGAGCTGCATTCTTTCCACC
AAAGCCAGCAACACTATAGCACCCTCCACAGAGTGGTGGACAATGGTCTGCAACTTA
GCCCTGAGGATGATGAGACGACAATGGTCCTTGTGGGTACTGCCCACAATATGTAGC
TCATCCACCATCAATGACTCATGGTGTTGAGTGGCATGCTCATTCTGTGATCCTCCTAACG
TATCACCAGCAACCTGTGTGTCTCTGTATTTTCTTACATTTGGAGTATGTTTACAAGACAAT
AACACAAAGGAAACTGCTTTGAAGCTCTCAAGTGGAA (SEQ ID NO 10)
h165-015D conceptual translation (SEQ ID NO 11)
MDAFQGJLKFFLNQKTVIGYSFMALLTVGSERLFSVVAFKCPCSTENMTYGLVFLFAPAWVTLL
ILGFFLNNRSWRLFTGCCVNPRKJFPRGHSCPYFYVLGQITLSSLVAPVMWLSVALLNGTFYEC
AMSGTRSSGLLELICKGKPKECWEELHKVSCGKTSMLPTVNEELKLSLQAQSQILGWCLICSA
SFFSLLTTCYARCRSKVSYLQLSFWKTYAQKEKEQLENTFLDYANKLSERNLKCFFENKRPDP
FPMPTFAAWEAASELHSFHQSQQHYSTLHRVVDNGLQLSPEDDETTMVLVGTAHNM
(SEQ ID NO 11)
h165-015E genomic DNA (SEQ ID NO 12)
h105-015E was identified from 6q22.1-22.33 BAC, accession number AL121953. Some intronic
sequence intervals are denoted as runs of N and predicted coding sequence is denoted in boldface.
TCAATCTGAGCTCAGGGTTTTCAAAGTACACAAGGAAAGATGCTAGAATCCTTTAAACAT
GTAAGGATGTTGGATCACTTGACACAACCAGTTAAACCAGTAAGATCCCATAAAATGGTT
ATAGCTGGTGGAGTCTAATGATCAGAAAGGGCCACAAGCTGATTTGTGTAACAGCTTCCC
AAGATGTGCCCAACTCTCAACAATATTGTGTCTTCTCTGCAGAGAAATGGAAATATTTA
TCAATTCTTTAATTGCAGCCTTGACTATTGGTGGGCAACAACTCTTCTCCTCTTCTAC
ATTCAGCTGTCCTTGTCAGGTTGGAAAAAATTTCTATTATGGTTCTGCTTTTCTTGTC
ATTCCTGCCTTGATCCTTCTCGTTGCTGGCTTTGCTCTGAGAAGCCAAATGTGGACA
ATTACCGGTGAATACTGCTGCAGCTGTGCCCCTCCATACAGGAGAATCAGCCCCCTA
GAGTGCAAGCTGGCTTGCCTTAGGTTCTTCAGCATCACTGGGAGGGCAGTTATTGCT
CCTTTAACTTGGCTGGCGGTGACCCTGCTGACAGGCACGTATTATGGTGTGCAGCA
AGTGAATTTGCATCTGTGGACCATTACCCAATGTTTGATAATGTCAGTGCCAGCAAA
CGAGAAGAGATCCTGGCTGGGTTTCCATGTTGCAGATCAGCTCCTTCTGACGTGATC
CTAGTAAGAGATGAAATAGCTCTTCTGCACAGATACCAGTCACAGGTAAGTTTTNNNN
NNNNNNNNNNNNNNNNNNNNNNNNNNNNNNNNNNNNNNNNNNNNNNATATTTTAGTTC
TTAGGTTTGTAGGAATCCCCCCTCCCATOGCTGTTGCTTGAATAGATGCATTGATACTT
CCTGTTTTTCTTTTTAATAATGATGTGAAGATGCTGGGTTGGATTTTGATCACCTTGGCA
ACCATTGCTGCCTTAGTCTCCTGCTGTGTGGCAAAGTGCTGCTCTCCCCTCACCTCTC
TGCAACATTGCTACTGGACCAGCCACCTCCAGAATGAGAGAGAACTCTTTGAACAAG
CAGCAGAGCAGCACTCTCGGCTCCTCATGATGCATCGCATAAAGAAAGCTATTTGGCT
TCATTCCCGGGAGTGAAGACGTCAAACACATCCGCATTCCTTCTTGTCAGGACTGGA
AAGATATTTCAGTACCCACTCTTTTATGCATGGGTGATGACTTGCAAGGTCACTATAG
CTTCCTTGGAAATAGGGTGGATGAGGATAATGAGGAGACAGATCAGAGGTATTGA
ATTAAAACCTTGATTACAGCACCIITCATGAGTCAGGCACTTAGCAGATACTTGGCTTTT
ATGGCTTTTAT (SEQ ID NO 12)
h165-015E conceptual translation (SEQ ID NO 13)
MCPTLNNIVSSLQRNGIFINSLIAALTIGGQQLFSSSTFSCPCQVGRNFYYGSAFLVIPALILLVA
GFALRSQMWTITGEYCCSCAYPYRPJSPLECKLACLRFFSITGRAVIAPLTWLAVTLLTGTYYE
CAASEFASVDHYPMFDNVSASKREEILAOFPCCRSAPSDVILVRDEIALLHRYQSQMLGWJLJT
LATIAALVSCCVAKCCSPLTSLQHCYWTSHLQNERELFEQAAEQHSRLLMMHRIKKFGFIPG
SEDVKHIRJPSCQDWKDISVPTLLCMGDDLQGHYSFLGNRVDEDNEEDRSRGIELLP
(SEQ ID NO 13)
h165-015F genomic DNA (SEQ ID NO 14)
h165-015F was identified from chr 10 HTGS, accession number AL139339. Some intronic sequence
intervals are denoted as runs of N and the predicted coding sequence is denoted in boldface.
GAGTCATGAGGTGGOCACCCAGTGGGCAGGGTGGOCAGCAGGGGCCCTCTTGGAGOCAG
CAGTGAGTTGGGAAGAGGAGGCCGGGCCCCACAGCGGGCATGATGGACAAGTTCCGGA
TGATCTTCCAGTTCCTGCAGTCCAACCAGGAGTCCTTCATGAATGGCATCTGTGGCA
TCATGGCCCTGGCCAGTGCCCAGATGTACTCGGCCTTCGACTTCAACTGCCCCTGCC
TGCCGGGCTACAATGCAGCCTACAGCGCGGGCATCCTGCTGGCGCCACCCCTGGTG
CTCTTTCTGCTTGGCCTGGTCATGAACAACAACGTGTCCATGCTGGCCGAAGAGTGG
AAGCGGCCGCTGGGCCGCCGGGCCAAGGACCCCGCTGTGTTGCGCTACATGTTCTG
CTCCATGGCCCAGCGCGCCCTCATCGCGCCTGTCGTCTGGGTGGCCGTCACGCTACT
CGACGGCAAATGCTTCCTCTGTGCCTTCTGCACTGCCGTGCCCGTGAGCGCACTGGG
CAACGGCAGCCTGGCACCCGGCCTTCCTGCCCCCGAGCTCGCCCGCCTGCTGGCCC
GGGTGCCCTGCCCTGAGATCTACGATGGCGACTGGCTGTTGGCCCGAGAGGTGGCC
GTGCGTTACCTCCGCTGCATCTCCCAGGTGAGGGGCCGCATGGCTTCACGCTGGGTCTC
CCTGGCAGATCAAGGTCCCTCTGGGAGGGCCCTATCCCCCTACCTCTCAAAATGGGGCCTC
TGCTNNNNNNNNNNNNNNNNNNNNNNNNNNNNNNTAGAGGCACTGGCATAGAGAGTGCTTT
GGGAGTCTGAACAGGCTCGGAGGCCCAAGGACAGTGGCCCGTGACTCTCTCTCATCTTCC
CACAGGCGCTGGGCTGGTCCTTCGTGCTGCTGACCACTCTGCTGGCATTCGTGGTGC
GCTCTGTGCGGCCCTGCTTCACGCAGGCCGCCTTCCTCAAGAGCAGTACTGGTCCC
ACTATATCGACATCGAGCGCAAGCTCTTCGACGAGACGTGCACGGAGCACGCCAAA
GCCTTTGCCAAGGTCTGCATCCAGCAGTTCTTCGAGGCCATGAACCATGACCTGGAG
CTGGGTCACACCCACGGGACACTGGCCACGGCCCCTGCTTCCGCAGCTGCCCCCAC
GACCCCCGATGGTGCGGAGGAGGAAAGGGAGAAGCTGCGTGGCATCACGGATCAAG
GCACCATGAACAGGCTGCTCACGAGCTGGCACAAATGCAAACCGCCTCTGCGGCTG
GGCCAGGAGGAGCCACCGCTGATGGGCAACGGCTGGGCTGGGGGTGGGCCCCGGC
CTCCGCGTAAGGAGGTGGCCACCTACTTCAGCAAAGTGTGAGGTGTGGCCAGCTGAA
GAGGCAGGAACGGGGATCTGAGCCCACAGCCCCTCCAACCCCCAAACCAGGTGGAAAAA
GGAAGGGTTTCAGTGCTGGGCAGTACTCCCCTAGGCAGATCCACACT (SEQ ID NO 14)
h165-015E conceptual translation (SEQ ID NO 15)
MDKFRMIFQFLQSNQESFMNGICGIMALASAQMYSAFDFNCPCLPGYNAAYSAGILLAPPLVL
FLLGLVMNNNVSMLAEEWKRPLGRRAKDPAVLRYMFCSMAQRALLAPVVWVAVTLLDGKC
FLCAFCTAVPVSALGNGSLAPGLPAPELARLLARVPCPEIYDGDWLLAREVAVRYLRCISQAL
GWSFVLLTTLLAFVVRSVRPCFTQAAFLKSKYWSHYTDIERKLFDETCTEHAKAFAKVCIQQFF
EAMNHDLELGHTHGTLATAPASAAAPTTPDGAEEEREKLRGITDQGTMNRLLTSWHKCKPPL
RLGQEEPPLMGNGWAGGGPRPPRKEVATTFSKV (SEQ ID NO 15)
EXAMPLE 3—mouse 165-015 TSTPs
m165-015C (mouse 2 TCP #3)conceptual translation (SEQ ID NO 16)
m165-015C was assembled from EST AA189546 and additional overlapping EST hits.
MEKEKAVLDLQMRKHRLGYSLVTLLTAGGEKIFSSVVEQCPCTATWNLPYGLLVLLVPALA
LFLLGYALSARTWRLLTGCCSRSARSSGLRSAFVCAQLSMTAAFAPLTWVAVALLEGSFYQC
AVSGSARLAPYLCKGRDPNCNATLPQAPCNKQKVEMQEILSQLKAQSQVFGWILIAAVIILLL
VKSVTRCFSPVSYLQLKFWEIYWEKEKQILQNQAAENATQLAEENVRCFFECSKPKECNTTSS
KDWQEISALYTFNPKNQFYSMLHKYVSREEMSGSVRSVEGDAVIPALGFVDDMSMTNTHEL
(SEQ ID NO 16)
m165-015D conceptual translation (SEQ ID NO 17)
m165-015D was assembled from EST A1181214.
AQGQAKECWEELHKVSCGKSSMAAMESEEVRLSLQAQSQILGWCLICSASFLSLLTTCYARC
RSKVSYLQLSFWKTYAQREKEQLENKLLECANKLSERNLKCFFENKKPDPFPMPSFGAWQHA
SELHSFHKDREHYSTLHKVVDDGMEQTPQEEETTMILVGTAQSL (SEQ ID NO 17)
EXAMPLE 4—pig 165-015 TSTIPs
p165-015B conceptual translation (SEQ ID NO 18)
p165-015B was assembled from pig EST AW416118.
PRVRKCLKHYCSPLSYRQEAYWTQYRTNEDQLFQRTAEVHSRVLAANNVRRFFGFVALDKD
DKELVAKFPVEGTQPRPQWNAITGVYLYRENQGLPLYSRLHKWAQGLAGNGTA
(SEQ ID NO 18)
EXAMPLE 5—bovine 165-015 TSTPs
b165-015B conceptual translation (SEQ lID NO 19)
b165-015B was assembled from cow EST AW353143.
GERSFPVAHATEILAREPCGEGPANLSVFREEVSRRLKYESQLFGWLLIGVVAILVFLTKCLKH
YCSPLSYRQEAYWAQYRANDDQLFQRTAEVHSRVLAANNVRPYEGFVALDDEEL
(SEQ ID NO 19)
EXAMPLE 6 - C. elegans 165-015 TSTPs
ce165-015 conceptual translation (SEQ ID NO 20)
C. elegans orf (open reading frame)
MTTSINSVVTVFQNVFTNHGSTLLNGILIATTVGGQSLVRKLTFSCPCAYPLNIYHSLVFMFGPT
AALLLIGITVNSTTWKLAHGFFFRVRDTRHISWKTTCVSWIEVLJQSSVAYIAWLFVVFLDGGY
YRCYRSHEFCLISDAILCKNSTTLNSYASTSSFNKISDNGKYCPPCICXTPNPTDASYLEAESQIYA
WGLLLFSGVAAFLVITCNRMCDKYTLVQRQYVETYKNVETQKDAVAKEHASQLAEHNARA
FFGQKDWTKDWDWVSGIPEPLFARLRLLIAAEKTQQTMYTPLQLWNDNKGYPJPQPDL
QLTQIIVDETKED (SEQ ID NO 20)
EXAMPLE 7- Human REPEATER TSTPs
Human REPEATER cds (first coding exon in boldface; second in normal
type) (SEQ ID NO 21
ATGCAGGGCCGCGTGGCAGGGAGCTGCGCTCCTCTGGGCCTGCTCCTGGTCTGTCT
TCATCTCCCAGGCCTCTTTGCCCGGAGCATCGGTGUGTGGAGGAGAAAGTTTCCCAAAA
CTTGGGGACCAACTTGCCTCAGCTCGGACAACCTTCCTCCACTGGCCCCTCTAACTCTGAA
CATCCGCACCCCGCTCTGGACCCTAGGTCTAATGACTTGGCAAGGOTTCCTCTGAAGCTCA
GCGTGCCTGCATCAGATGGCTTCCCACCTGCAGGACGTTCTGCAGTGCAGAGGTGGCCTC
CATCGTGGGGGCTGCCTGCCATGGATTCCTGGCCCCCTGAGGATCCTTGGCAGATGATGGC
TGCTCCGGCTGAGGACCGCCTGGGGGAAGCGCTGCCTGAAGAACTCTCTTACCTCTCCAG
TGCTGCGGCCCTCGCTCCGGGCAGTGGCCCTTTGCCTGGGGAGTCTTACCCGATGCCACA
GGCCTCTCACCCGAGGCTTCACTCCTCCACCAGGACTCGGAGTCCAGACGACTGCCCCGTT
CTAATTCACTGGGAGCCGGGGGAAAAATCCTTTCCCAACGCCCTCCCTGGTCTCTCATCCA
CAGGGTTCTGCCTGATCACCCCTGGGGTACCCTGAATCCCAGTGTGTCCTGGGGAGGTGG
AGGCCCTGGGACTGGTTGGGGAACGAGGCCCATGCCACACCCTGAGGGAATCTGGGGTAT
CAATAATCAACCCCCAGGTACCAGCTGGGGAAATATTAATCGGTATCCAGGAGGCAGCTG
GGGAAATATTAATCGGTATCCAGGAGGCAGCTGGGGGAATATTAATCGGTATCCAGGAGG
CAGCTGGGGGAATATTCATCTATACCCAGGTATCATTAACCCATTTCCTCCTGGAGTTCTC
CGCCCTCCTGGCTCTTCTTGGAACATCCCAGCTGGCTTCCCTAATCCTCCAAGCCCTAGGTT
GCAGTGGGGCTAG (SEQ ID NO 21)
Human REPEATER conceptual translation (SEQ ID NO 22)
MQGRVAGSCAPLGLLLVCLHLPGLFARSIGVVEEKVSQNLGTNLPQLGQPSSTGPSNSEHPQP
ALDPRSNDLARVPLKLSVPASDGFPPAGGSAVQRWPPSWGLPAMDSWPPEDPWQMMAAAAE
DRLGEALPEELSYLSSAAALAPGSGPLPOESSPDATGLSPEASLLHQDSESRRLPRSNSLGAGG
KILSQRPPWSLIHRVLPDHPWGTLNPSVSWGGGGPGTGWGTRPMPHPEOIWGINNQPPGTSW
PNPPSPRLQWG (SEQ ID NO 22)
EXAMPLE 8-Human LUNCH TSTPs
Human LUNCH gene fragment (coding exons in boldface) (SEQ ID NO 23)
TAGGGCAGGAGGGAAGGTGGGGGGTGGCACGTGCCGGCCTTCCATGCCTCTGCCCATCCT
CAGCTCCAGCCCCTCTACCACGAGGCCTTCCCCGGCTTCCAGGGCATCGGTGTCCTG
GTCTTCAGGTGAGTGCACGTGGCTCTCAGGGCCCACCCATCACCCACCAGCTGCTCTGAC
CCTTCTCAGCCACAAAGGCACTGTCCCAGATGCCTAGCTCTGCCCGTCCCACCCCCTGTTC
AGGAGCACCTGGGGACAGAGGCAGGAAGAGCCCTGGACAOGCAGGGAGGAGGCCCACGT
CTGATTCTGCCACTGGCTATGCTGTGTGACCTCATATGCCCTTTGGCCTGCCCTGAGCCCTG
ATTCCAGCTGCAGGATGTGGGCAGGAACATCAGGCATTGTCTGAGTGCAGTGGGGAAGGC
AGAGGCAGCAAGGGCAGCAGGCTTGTAAATGACATGCAAAGGGATGCAATCCCGCTTGGG
CAGGGCCCTCCACTCTAAGCGTCTGGGGGAAGACGATGTTGAGGGAGACCGAGACCATAT
TTGCCAGCGGAGAGCAGCCCTGCCATGTCATGAGAAAGGCTGAGAAGGTCCAAATCACTG
CAGCCCCACTTGAGTTGTGAOCTCACTGTGGGCTTTGAATCCTGTAAGTTTAAAGGCT
CAAATGCTCCCAGTGACAGGAAGCTCACTCTCTCTCCAGGCCATGCTCCTGAGCTGCTCAG
ACAGTTTTCCCTTTTAATAAATGGAAGCCTTCTATGACTTCCCTGCCCTGGGCCTGAAGAG
AAGTTTTCTCTCCCCACCCCCAAGATGCACAGCACGCAAGTGCTCAGGGGGAGGACAAAA
GTTATTCTGGATTCTTATTTGTTTCCTGGTCTCTCCCCGAAAGCTCCTTCAGTGCCTTACTG
AGGCCAGCGTACCTCCTCAGGCCATGGGCAATTGGTCCATCTCCCACTGGCAATCAGAGAT
TCTTCAGGCCTGTCATCTCCCTCTCTCCAGAGCCCCTGCTCCTTGTGATGCAGCCGAGAT
GTTTTTCTCAGTGTGACCCAGATTATCACCCTGAGAGCTCACAGCCAGCTGCAGCCTGGCT
GAGTAACCCTGCCCTGGGAGCTCCCTGGGTGGGAHCATGGTATTCCCATGACAGCCGGCT
TGTTGGGGTGTCCAAGGGAACTCAGTTTCACTGAGTGCCTGCTTTGGATGCTGGTGGCTGA
GTCATTCAGGCCTCACAGCAATCCTCTGGGAAAGGGATCATTATCTCCCTTTACTGAGGA
GGCAGCCAAGGCCCAGACGGGTGAGGTGACTTGCCTTGAGTTACACAGGGAGGCTCTTCT
CTGCGTGACCCATGACTTCCCCTGTGGACTTCCCTCCCTTGGGATGCCACTCGTGCACCA
GCCTGGCCCCCACCGGGTGCACAGGACCCCTCATGCCCCCACTGGCCCTGGCTGAGGACG
TGGCTCTGCCCCCACAGGCCTGGCTCTGTAGCGGTGAACGCCTCCCTTGTATTTGGGG
GCCGTGCCCCAGGCCCCTCTCCCTGTGAACTCCTCTGGGCTTTGTATCGCAAAGTGA
AGACCTCAGGGCACATGCTGGGGAACCTCTCATTGGCTGAGAACAGCCTCACCTCTG
ATGGTGAGTCCCATCCCCAGCCGCCCCTACCGCAGTGCCTTTGACCTCCCCAGGGGGAGC
ACTGGGTGGACTTCCTGGAGGGATCCAACTTCTGCCCTGACCCCGAAGCACCTGGTGGTG
CGGTGGGCAAOOAGGGTCTTGGCGCCTCCGGAACTCTCACCCATGGCTCTACAGGGGCCG
ACTTGATCACCCTGGCCCGGGAGACCATCAGCATCCGCTTCACAGCCATGAGGTCCT
TCCTGCCGCAGCTCCTCGTACCGGGTTCTGTTTCCTTTGTCCTGCTGGAAAGGCAGA
TCCTCCAACAGGTGAGGTGGCATAGTGAGGACACTCACTATACCTCCCTTGAAGCAGGCC
TCCCAGCATGGAGGCAOCACCCAGCCATGGCACTATGGCGGCGGCAGTCATAGAGGGC
ACCCTCGGGGAGAGGGTTCTCTGAAGGAGGAGGTGACTGTCCGAATGGGAACACAGCCA
GGCTCTGTAAGCAGACACTCCTACCCAGGACGCCGTGTTTAAGGGTGCGCTCCCTCCCAG
GTCCTGCCTCCCACCTCACCTGGTCCCCATCCCACACTGGGTCTCAGGAGATACAGCTCCC
AGGCCCGCCCTGACCTCCAGGGTTTCCCACAGGTCACACCGGTGGTGTCAGGATTCTAC
AAGGCGAGTCCCCAGGAGAGGCCCCTGCTCCTCTTCAGGTGGGTCGAGTCCCCCTCCC
ATCCACTCAGCCTGCCCTGCTGCTCTCTTGGGGGCTGCCCTGGGACAGAGGGAGGCAGCT
CTGTGACCCAGAACCAGGATGTGGGGGTGTAGGTTTGGGGCTAGCTGGGAAAGGACTTTG
CCCCAGGTAGCTGTAGCTTGGCTGTGTAAGCTAGCTCTGCCTAGAAGGTAGGGGGCCCCC
ACGCCAGAGGGTGTGCATTTTGGGGGACTCTACCTGCTCTAGAAACGTGCTGGCAAAGAG
CTTGTGACCTCTGCACTCTCCCCTTGTGCTTCTCTAGCAATGCGGACCAGTGGGTGGGTG
TTTATATCGAATACAAGTTCCAGACTCCCATCACTACCCACCTCCAAGGCCTGGCTAA
TCACTTGGCCCAAACATAACAGATCCCATCCTCCAGAAATCCAGCATCGTGGCCAA
TGGTGAGTCGGGGCTGCAATCCCTGTGTGTCTCTGGGCCAGCAGGCTCTTTCCCTCTCTAA
ACCTTGGTGTTCCCCTTCTAGAGAATGGGCAGAATTCCTTCAGACAACACTTGCTCATGT
GTTGGGGCCACAGAGAGGAATGGGGCATAGAGCTTTAGCAAGGAGCTGGACAGAGGCGA
GAAGCAGTCAAGATCAGAAGGATGAGAGGGTCCCGTCACCTCCCTGATTGCCCAGGACCT
CACTGAGTCCTCACATGCATGACAAGATCTGTCCCACCAAGTCAGGGGCAAAAGCCCTCA
GCTCTGCCCCAGTGCAGGGGCTGCAAGAAGCCCATATCTCCTCTTGGGGCCTCCTGCAGG
ATGACTTCTGCAAGCTTGTTGCCATTCAAACAGGGAGAGCTGTTCCACACCCAAGTTAGG
AGAGCGCTGGGTCAAATCAGTCCCCTCCAGCTACGCACAGCCCAGAGCTGCTCTCCGAGG
ACCCCTCAGCCACAGAAGGTGCTGGTCCTCCTACAGTGCTGCCCCACCCCCACCACCACA
GCCTCCTGGGGGTGCGGTCGGGGGTGCGTTCAGGGGTGCCCTCCTTCGGGCTTTGCCCCTG
CACTCACTCCTACACAACTACTCTCCCTGCCGGCAGCCCCAGAGACCAGATTGGTTTGGGC
CTCTGCCTCCGCAGGCCCGAACCACCATCTCTTCTGCTCCCCAGAAAGCCAGCTCCCAGCA
CACAGGCAGCGTTCCCTCTTCATTCCTCAACACAGAGGGCCCCTCACAACCCTCTCAACAG
ATGGTAGAGCTCCCCATGTGTCCTTAAAGAGGCTTCACCATTGATGGCCACAAGCCCCA
CCATGACCTCCACCCAGGGAGTGAATTTTCTTACGTGTCTCCCACCGGATCAGGGACAAA
ATAAAACATAAAAGCAGCAGCTGTTAAATTATCAGCAACATTTCCTACTAGGATCAACTC
ACGGAGTGGGCAGTGTATTCCATTAGATGGATTAGTACAGGGGTTAGCAGCATTTTCTGT
AAAGGGCCCGATGGGAACTCTTTAAGGCTCTGCAGGCCACTGGGTCTCTGTCACCACCTA
CTCAAACCCTCCTCTTAGCAAGAAAGCAGCCCTAGGCAACATGTCAACAATGAGCGTGG
CCACGTTCCCGGAAACCTTTCTTTATGGACATGGAAGTTTGCATCTCACGTACTTCTCATGT
GTCACAAGATACGATTCTACTTTCCATGPITTCAACCATTTAAACATGTAAAAGCCATTCT
TAATTCATGGGCCACACAAAAACAGGCAOCAAGAAATTTGGTTTTTAGACCAGTGGCAGC
TGACCCCTGGATGGAGCATAAGAGCTTGGGTGTGTGTCCCACTCCAGGGCTTCCAGGAGA
GGGGATGCAGGCTGATGCCACCTCCACCCCCATCACTCTAGGGGAGAAGGCAGAGCTGG
TGCTGTATGAAGTTTGGCTGCAGATCCTGGTCCAGCCCTACACCAAGGCTTTGGAGG
ACAAAACCAGCCCTGAGTTCTGGGCACTTCAAGGGCAGCTGACGAGATGGGTGAAGT
GGCGGGATGAAGTGGGGGGTCGAGGAAGAGGCCTCGGGAAGATGGGGGATGACTGGACA
AGACTTTGGTAGAGCTGTGACCCTCYrCATCCTCTCCTGACCCCCCAGCTGAACTTCATCC
TCAGACCTCTGCAGAACTTTGACCAAGTGGTGGTGGAGGAATTCCCGTGGGTTCAGG
GTGGCCCTGGGTGACCCAGGGCTCAGACCTGGGTTCTGOGGTTGAGGGCAGGAGGCTGGA
AGAGATGACCTCCCTAGTCCCCCAAGGCTGCCCAGCCCCCTGAAGTCTGTGATGATCCTC
CCACCAGGCCGGAGCCACTGACTGCCAGAATGGGTGCCACCTTCTTCAGGGCGGCGC
CAGCCCAGGCTCTCATGTGGGACCGTTTGCGCCAGGGTCTGCACACCCTGGGGAAG
GCAGAGGGCCTCTTGGTGGAGATGGTCATCCCAGACCTCGGTCAGTACCTCCTTCCCT
CTGCAGGCCCCCTCCCTCTGTCCATCTGCCTCCTCCCTTGGGCTCGCTGCCTCCACATGCCC
TGATCTGAAGCCTGCCTCCCCTCCTCATGGAGCCCTCCAAGGTGCTCCTAGCCCCAGCTCC
CTGGTCCCGCAGCACCTTCTGAGCCCAGATTCTGCCTTCTCAGAAGTCTGGGAGGCAGGGC
CCCGCCTGGCCATGGCCTTCTTGCTCTGCTCCAGGC (SEQ ID NO 23)
Human LUNCH partial conceptual translation (SEQ ID NO 24)
LQPLYHEAFPGFQGIGVLVFRPGSVAVNASLVFGGRAPGPSPCELLWALYRKVKTSGHMLGN
LSLAENSLTSDGADLITLARETISIRFTAMRSFLPQLLVPGSVSFVLLERQILQQVTPVVSGFYKA
SPQERPLLLFSNADQWVGVYIEYKFQTPTTTHLQGLANHLAQNITDPILQKSSIVANGEKAELV
LYEVWLQILVQPYTKALEDKTSPEFWALQGQLTRWLNFILRPLWNFDWVVVEEFPPEPLTARM
GATFFRAAPAQALMWDRLRQGLHTLGKAEGLLVEMVIPDL (SEQ ID NO 24)

While the foregoing detailed description has described several embodiments of the present invention, it is to be understood that the above description is illustrative only and not limiting of the disclosed invention. The invention is to be limited only by the claims which follow.

What is claimed:

1. An isolated nucleic acid molecule comprising a nucleic acid sequence selected from the group consisting of:
(i) a nucleic acid sequence coding for a TSTP comprising a nucleic acid sequence of SEQ ID NOS: 4, 6, 10, 12, 14, 21, and 23;
(ii) a nucleic acid sequence coding for a TSTP, wherein the TSTP comprises an amino acid sequence selected from the group consisting of SEQ ID NOS: 2, 3, 5, 7, 11, 13, 15, 17, 18, 19, 20, 22, 24, and conservatively modified variants thereof;
(iii) a nucleic acid sequence coding for a TSTP having at least about 75% nucleic acid sequence identity to the TSTP encoding regions of SEQ ID NOS: 4, 6, 10, 12, 14, 21, and 23;
(iv) a nucleic acid sequence having at least about 75% sequence identity to a sequence encoding a TSTP, wherein the polypeptide comprises an amino acid sequence selected from the group consisting of SEQ ID NOS: 2, 3, 5, 7, 11, 13, 15, 17, 18, 19, 20, 22, 24; and conservatively modified variants thereof, and
(v) a variant of a nucleotide sequence selected from the group consisting of SEQ ID NOS: 4, 6, 10, 12, 14, 21, and 23, containing at least one conservative substitution in a region coding for a TSTP.

2. An isolated nucleic acid molecule consisting essentially of a nucleic acid sequence selected from the group consisting of:
(i) a nucleic acid sequence coding for a TSTP comprising a nucleic acid sequence of SEQ ID NOS: 4, 6, 10, 12, 14, 21, and 23;
(ii) a nucleic acid sequence coding for a TSTP, wherein the TSTP comprises an amino acid sequence selected from the group consisting of SEQ ID NOS: 2, 3, 5, 7, 11, 13, 15, 17, 18, 19,20,22,24, and conservatively modified variants thereof;
(iii) a nucleic acid sequence coding for a TSTP having at least about 75% nucleic acid sequence identity to the TSTP encoding regions of SEQ ID NOS: 4, 6, 10, 12, 14, 21, and 23;
(iv) a nucleic acid sequence having at least about 75% sequence identity to a sequence encoding a TSTP, wherein the polypeptide comprises an amino acid sequence selected from the group consisting of SEQ ID NOS: 2, 3, 5, 7, 11, 13, 15, 17, 18, 19, 20, 22, 24; and conservatively modified variants thereof; and
(v) a variant of a nucleotide sequence selected from the group consisting of SEQ ID NOS: 4, 6, 10, 12, 14, 21, and 23, containing at least one conservative substitution in a region coding for a TSTP.

3. An isolated nucleic acid molecule consisting essentially of a nucleic acid sequence coding for a TSTP selected from the group consisting of:
(i) a cDNA molecule consisting essentially of a nucleic acid sequence coding for a TSTP, wherein said nucleic acid sequence comprises the sequence of SEQ ID NO: 21; and
(ii) an isolated genomic DNA sequence consisting essentially of a sequence coding for a TSTP, wherein said nucleic acid sequence comprises the sequence of SEQ ID NOS: 4, 6, 10, 12, 14, and 23.

4. An isolated RNA transcribed from an isolated nucleic acid molecule according to claim 3.

5. An isolated nucleic acid molecule that hybridizes to a nucleic acid molecule according to claim 3 under stringent hybridization conditions.

6. An isolated nucleic acid molecule that hybridizes to a nucleic acid molecule according to claim 3 under moderate hybridization conditions.

7. An isolated fragment of a nucleic acid molecule according to claim 3 that is at least about 20 to 30 nucleotide bases in length.

8. A chimeric or fused nucleic acid molecule, wherein said chimeric or fused nucleic acid comprises at least part of a TSTP coding sequence contained in an isolated nucleic acid molecule according to claim 3, and at least part of a heterologous coding sequence, wherein transcription of said chimeric or fused nucleic acid results in a single chimeric nucleic acid transcript.

9. The chimeric or fused nucleic acid molecule of claim 8, wherein said heterologous coding sequence is a sequence that facilitates expression of all or part of said TSTP.

10. The chimeric or fused nucleic acid molecule of claim 9, wherein said heterologous coding sequence is from a detectable marker gene.

11. The chimeric or fused nucleic acid of claim 10, wherein said heterologous coding sequence is from a gene encoding green fluorescence protein.

12. An isolated nucleic acid molecule having at least about 75% identity to a nucleic acid sequence coding for a TSTP selected from the group consisting of:
(i) a cDNA molecule consisting essentially of a nucleic acid sequence coding for a TSTP, wherein said nucleic acid sequence comprises the sequence of SEQ ID NO: 21; and
(ii) an isolated genomic DNA sequence consisting essentially of a sequence coding for a TSTP, wherein said nucleic acid sequence comprises the sequence of SEQ ID NOS: 4, 6, 10, 12, 14, and 23.

13. An isolated RNA transcribed from a nucleic acid molecule according to claim 12.

14. An isolated nucleic acid that hybridizes to a nucleic acid molecule according to under stringent hybridization conditions.

15. An isolated nucleic acid that hybridizes to a nucleic acid molecule according to under moderate hybridization conditions.

16. An isolated fragment of a nucleic acid molecule according to that is at least about 20 to 30 nucleotide bases in length.

17. A chimeric or fused nucleic acid molecule, wherein said chimeric or fused nucleic acid comprises at least part of the TSTP coding sequence of a nucleic acid molecule according to claim 12, and at least part of a heterologous coding sequence, wherein transcription of said chimeric or fused nucleic acid results in a single chimeric nucleic acid transcript.

18. The chimeric or fused nucleic acid molecule of claim 17, wherein said heterologous coding sequence is from a sequence encoding a TSTP paralog or ortholog.

19. The chimeric or fused nucleic acid of claim 18, wherein said heterologous coding sequence is a sequence that facilitates expression of all or part of said TSTP.

20. The chimeric or fused nucleic acid of claim 18, wherein said heterologous coding sequence is from a detectable marker gene.

21. The chimeric or fused nucleic acid of claim 20, wherein said heterologous coding sequence is from a gene encoding green fluorescence protein.

22. An isolated genomic DNA molecule consisting essentially of a sequence coding for a TSTP having an amino acid sequence that is at least about 75% identical to the amino acid sequence selected from the group consisting of SEQ ID NOS: 2,3,5,7, 11, 13, 15, 17, 18, 19,20,22, and 24.

23. An isolated RNA transcribed from the isolated DNA of claim 22.

24. An isolated nucleic acid that hybridizes to the DNA of claim 22 under stringent hybridization conditions.

25. An isolated nucleic acid that hybridizes to the DNA of claim 22 under moderate hybridization conditions.

26. An isolated fragment of the nucleic acid of claim 22 that is at least about 20 to 30 nucleotide bases in length.

27. A chimeric or fused nucleic acid, wherein said chimeric or fused nucleic acid comprises at least part of the coding sequence contained in the isolated DNA sequence of claim 22 and at least part of a heterologous coding sequence, wherein transcription of said chimeric or fused nucleic acid results in a single chimeric nucleic acid transcript.

28. The chimeric or fused nucleic acid of claim 27, wherein said heterologous coding sequence is from a sequence encoding a TSTP paralog or ortholog.

29. The chimeric or fused nucleic acid of claim 27, wherein said heterologous coding sequence is a sequence that facilitates expression of all or part of said TSTP.

30. The chimeric or fused nucleic acid of claim 27, wherein said heterologous coding sequence is from a detectable marker gene.

31. The chimeric or fused nucleic acid of claim 30, wherein said heterologous coding sequence is from a gene encoding green fluorescence protein.

32. An isolated cDNA molecule comprising a nucleic acid sequence selected from the group consisting of:
(i) the nucleic acid sequence of SEQ ID NO 21 coding for a REPEATER polypeptide;
(ii) a nucleic acid sequence having the same sequence as the region coding a LUNCH polypeptide contained in the genomic DNA sequence of SEQ ID NO 23; and
(iii) a nucleic acid sequence having the same sequence as the region coding a 165-015 polypeptide contained in the genomic DNA sequence of SEQ ID NOS: 4, 6, 10, 12, and 14.

33. An isolated RNA transcribed from the isolated cDNA of claim 32.

34. An isolated nucleic acid that hybridizes to the cDNA of claim 32 under stringent hybridization conditions.

35. An isolated nucleic acid that hybridizes to the cDNA of claim 32 under moderate hybridization conditions.

36. An isolated fragment of the cDNA of claim 32 that is at least about 20 to 30 nucleotide bases in length.

37. A chimeric or fused nucleic acid, wherein said chimeric or fused nucleic acid comprises at least part of the coding sequence contained in the isolated cDNA sequence of claim 32 and at least part of a heterologous coding sequence, wherein transcription of said chimeric or fused nucleic acid results in a single chimeric nucleic acid transcript.

38. The chimeric or fused nucleic acid of claim 37, wherein said heterologous coding sequence is from a sequence encoding a REPEATER, LUNCH, or 165-015 paralog or ortholog.

39. The chimeric or fused nucleic acid of claim 37, wherein said heterologous coding sequence is a sequence that facilitates expression of all or part of said REPEATER, LUNCH, or 165-015 polypeptide.

40. The chimeric or fused nucleic acid of claim 37, wherein said heterologous coding sequence is from a detectable marker gene.

41. The chimeric or fused nucleic acid of claim 40, wherein said heterologous coding sequence is from a gene encoding green fluorescence protein.

42. A nucleic acid molecule comprising the isolated cDNA of claim 32 operably linked to a heterologous promoter that is either regulatable or constitutive.

43. The nucleic acid molecule of claim 42, wherein said regulatable promoter is inducible under specific environmental or developmental conditions.

44. An isolated cDNA sequence coding for a protein having an amino acid sequence selected from the group consisting of SEQ ID NOS: 2, 3, 5, 7, 11, 12, 15, 17, 18, 19,20,22, and 24.

45. An isolated RNA transcribed from the isolated cDNA of claim 44.

46. An isolated nucleic acid that hybridizes to the cDNA of claim 44 under stringent hybridization conditions.

47. An isolated nucleic acid that hybridizes to the cDNA of claim 44 under moderate hybridization conditions.

48. An isolated fragment of the cDNA of claim 44 that is at least about 20 to 30 nucleotide bases in length.

49. A chimeric or fused nucleic acid, wherein said chimeric or fused nucleic acid comprises at least part of the coding sequence contained in the isolated cDNA sequence of claim 44 and at least part of a heterologous coding sequence, wherein transcription of said chimeric or fused nucleic acid results in a single chimeric nucleic acid transcript.

50. The chimeric or fused nucleic acid of claim 49, wherein said heterologous coding sequence is from a sequence encoding a TSTP paralog or ortholog.

51. The chimeric or fused nucleic acid of claim 49, wherein said heterologous coding sequence is a sequence that facilitates expression of all or part of said TSTP.

52. The chimeric or fused nucleic acid of claim 49, wherein said heterologous coding sequence is from a detectable marker gene.

53. The chimeric or fused nucleic acid of claim 52, wherein said heterologous coding sequence is from a gene encoding green fluorescence protein.

54. A nucleic acid comprising the isolated cDNA of claim 44 operably linked to a heterologous promoter that is either regulatable or constitutive.

55. The nucleic acid of claim 54, wherein said regulatable promoter is inducible under specific environmental or developmental conditions.

56. An isolated cDNA sequence having at least about 75% sequence identity to a nucleic acid sequence selected from the group consisting of:
(i) the nucleic acid sequence of SEQ ID NO 21 coding for a REPEATER polypeptide;
(ii) a nucleic acid sequence having the same sequence as the region coding a LUNCH polypeptide contained in the genomic DNA sequence of SEQ ID NO 23; and
(iii) a nucleic acid sequence having the same sequence as the region coding a 165-015 polypeptide contained in the genomic DNA sequence of SEQ ID NOS: 4, 6, 10, 12, and 14.

57. An isolated RNA transcribed from the isolated cDNA of claim 56.

58. An isolated nucleic acid that hybridizes to the cDNA of claim 56 under stringent hybridization conditions.

59. An isolated nucleic acid that hybridizes to the cDNA of claim 56 under moderate hybridization conditions.

60. An isolated fragment of the nucleic acid of claim 56 that is at least about 20 to 30 nucleotide bases in length.

61. A chimeric or fused nucleic acid, wherein said chimeric or fused nucleic acid comprises at least part of the coding sequence contained in the isolated cDNA sequence of claim 56 and at least part of a heterologous coding sequence, wherein transcription of said chimeric or fused nucleic acid results in a single chimeric nucleic acid transcript.

62. The chimeric or fused nucleic acid of claim 61, wherein said heterologous coding sequence is from a sequence encoding a REPEATER, LUNCH, or 165-015 paralog or ortholog.

63. The chimeric or fused nucleic acid of claim 61, wherein said heterologous coding sequence is a sequence that facilitates expression of all or part of said REPEATER, LUNCH, or 165-015 polypeptide.

64. The chimeric or fused nucleic acid of claim 61, wherein said heterologous coding sequence is from a detectable marker gene.

65. The chimeric or fused nucleic acid of claim 64, wherein said heterologous coding sequence is from a gene encoding green fluorescence protein.

66. A nucleic acid comprising the isolated cDNA of claim 56 operably linked to a heterologous promoter that is either regulatable or constitutive.

67. The nucleic acid of claim 66, wherein said regulatable promoter is inducible under specific environmental or developmental conditions.

68. An isolated cDNA sequence having at least about 75% sequence identity to a sequence encoding a TSTP having an amino acid sequence selected from the group consisting of SEQ ID NOS: 2, 3, 5, 7, 11, 12, 15, 17, 18, 19, 20, 22, and 24.

69. An isolated RNA transcribed from the isolated cDNA of claim 68.

70. An isolated nucleic acid that hybridizes to the cDNA of claim 68 under stringent hybridization conditions.

71. An isolated nucleic acid that hybridizes to the cDNA of claim 68 under moderate hybridization conditions.

72. An isolated fragment of the cDNA of claim 68 that is at least about 20 to 30 nucleotide bases in length.

73. A chimeric or fused nucleic acid, wherein said chimeric or fused nucleic acid comprises at least part of the coding sequence contained in the isolated cDNA sequence of claim 68 and at least part of a heterologous coding sequence, wherein transcription of said chimeric or fused nucleic acid results in a single chimeric nucleic acid transcript.

74. The chimeric or fused nucleic acid of claim 73, wherein said heterologous coding sequence is from a sequence encoding a TSTP paralog or ortholog.

75. The chimeric or fused nucleic acid of claim 73, wherein said heterologous coding sequence is a sequence that facilitates expression of all or part of said TSTP.

76. The chimeric or fused nucleic acid of claim 73, wherein said heterologous coding sequence is from a detectable marker gene.

77. The chimeric or fused nucleic acid of claim 76, wherein said heterologous coding sequence is from a gene encoding green fluorescence protein.

78. A nucleic acid comprising the isolated cDNA of claim 68 operably linked to a heterologous promoter that is either regulatable or constitutive.

79. The nucleic acid of claim 78, wherein said regulatable promoter is inducible under specific environmental or developmental conditions.

80. An isolated variant of a nucleic acid sequence selected from the group consisting of SEQ ID NOS: 4, 6, 10, 12, 14, 21, and 23, containing at least one conservative substitution in a region coding for a TSTP.

81. An isolated RNA transcribed from the isolated variant of claim 80.

82. An isolated nucleic acid that hybridizes to the variant of claim 80 under stringent hybridization conditions.

83. An isolated nucleic acid that hybridizes to the variant of claim 80 under moderate hybridization conditions.

84. An isolated fragment of the variant of claim 80 that is at least about 20 to 30 nucleotide bases in length.

85. A chimeric or fused nucleic acid, wherein said chimeric or fused nucleic acid comprises at least part of the coding sequence contained in the isolated variant of claim 80 and at least part of a heterologous coding sequence, wherein transcription of said chimeric or fused nucleic acid results in a single chimeric nucleic acid transcript.

86. The chimeric or fused nucleic acid of claim 85, wherein said heterologous coding sequence is from a sequence encoding a TSTP paralog or ortholog.

87. The chimeric or fused nucleic acid of claim 85, wherein said heterologous coding sequence is a sequence that facilitates expression of all or part of said TSTP.

88. The chimeric or fused nucleic acid of claim 85, wherein said heterologous coding sequence is from a detectable marker gene.

89. The chimeric or fused nucleic acid of claim 88, wherein said heterologous coding sequence is from a gene encoding green fluorescence protein.

90. A cDNA having the same nucleic acid sequence as the coding region of the variant of claim 80.

91. A nucleic acid comprising the cDNA of claim 90 operably linked to a heterologous promoter that is either regulatable or constitutive.

92. The nucleic acid of claim 91, wherein said regulatable promoter is inducible under specific environmental or developmental conditions.

93. An isolated variant of a nucleic acid sequence encoding a TSTP having an amino acid sequence selected from the group consisting of SEQ ID NOS: 2, 3, 5, 7, 11, 13, 15, 17, 18, 19, 20, 22, and 24, containing at least one conservative substitution in a TSTP coding region.

94. An isolated RNA transcribed from the isolated variant of claim 93.

95. An isolated nucleic acid that hybridizes to the variant of claim 93 under stringent hybridization conditions.

96. An isolated nucleic acid that hybridizes to the variant of claim 93 under moderate hybridization conditions.

97. An isolated fragment of the variant of claim 93 that is at least about 20 to 30 nucleotide bases in length.

98. A chimeric or fused nucleic acid, wherein said chimeric or fused nucleic acid comprises at least part of the coding sequence contained in the isolated variant of claim 93, and at least part of a heterologous coding sequence, wherein transcription of said chimeric or fused nucleic acid results in a single chimeric nucleic acid transcript.

99. The chimeric or fused nucleic acid of claim 98, wherein said heterologous coding sequence is from a sequence encoding a TSTP paralog or ortholog.

100. The chimeric or fused nucleic acid of claim 98, wherein said heterologous coding sequence is a sequence that facilitates expression of all or part of said TSTP.

101. The chimeric or fused nucleic acid of claim 98, wherein said heterologous coding sequence is from a detectable marker gene.

102. The chimeric or fused nucleic acid of claim 101, wherein said heterologous coding sequence is from a gene encoding green fluorescence protein.

103. A cDNA having the same nucleic acid sequence as the coding region of the variant of claim 93.

104. A nucleic acid comprising the cDNA of claim 103 operably linked to a heterologous promoter that is either regulatable or constitutive.

105. The nucleic acid of claim 104, wherein said regulatable promoter is inducible under specific environmental or developmental conditions.

106. The isolated nucleic acid of claim 1, wherein said nucleic acid encodes a REPEATER, LUNCH, or 165-015 polypeptide

107. An expression vector comprising the isolated nucleic acid of claim 1, wherein said vector is selected from the group consisting of vectors, bacterial plasmids, bacterial phagemids, viruses and retroviruses, bacteriophage vectors and linear or circular DNA molecules capable of integrating into a host cell genome.

108. A host cell transfected with the expression vector of claim 107, wherein said host cell expresses a TSTP.

109. A nucleic acid array comprising at least about 20 to 30 nucleotides of the isolated nucleic acid of claim 1, wherein said nucleic acid is linked covalently or noncovalently to a solid phase support.

110. A method of screening for compounds that activate TSTP-related signal transduction comprising:
(i) contacting the host cell of claim 108 with a putative signal activating compound; and
(ii) measuring activity of signal transduction associated with a TSTP expressed in said cell.

111. The method of claim 110, wherein said TSTP is a human polypeptide.

112. The method of claim 110, wherein said host cell is transfected with at least one additional nucleic acid construct encoding a gene involved signal transduction.

113. The method of claim 112, wherein said at least one additional gene encodes a G Protein-Coupled Receptor involved in taste signal transduction.

114. The method of claim 113, wherein said cell further expresses a Gα15 protein or other promiscuous G protein.

115. A method of screening for compounds that modulate taste signaling transduction comprising:
(i) contacting a host cell according to claim 108 with a known taste activating compound and a compound putatively involved in taste transduction modulation;
(ii) contacting a host cell according to claim 108 with a known taste activating compound alone; and
(iii) comparing signal transduction activity associated with the host cell of step (i) with the activity from the host cell of step (ii) to identify modulators of taste signal transduction.

116. The method of claim 115, wherein said modulatory compounds are selected from the group consisting of activators, inhibitors, stimulators, enhancers, agonists and antagonists.

117. The method of claim 115, wherein said TSTP is human.

118. The method of claim 115, wherein said host cell is transfected with at least one additional nucleic acid construct encoding a gene involved in signal transduction.

119. The method of claim 118, wherein said at least one additional gene encodes a G Protein-Coupled Receptor involved in taste signal transduction.

120. The method of claim 118, wherein said at least one additional protein is a Gα15 protein or other promiscuous G protein.

121. A method of detecting expression of a TSTP gene in a cell comprising:
(i) contacting said cell with a nucleic acid that hybridizes to the isolated nucleic acid of claim 1 under stringent conditions; and
(ii) detecting hybridization in order to detect expression of said TSTP gene.

122. A method of detecting expression of a TSTP gene in a cell comprising:
(i) contacting said cell with a nucleic acid that hybridizes to the isolated nucleic acid of claim 1 under moderate conditions; and
(ii) detecting hybridization in order to detect expression of said TSTP gene.

123. An isolated nucleic acid having the nucleotide sequence of SEQ ID NO: 4.

124. An isolated nucleic acid having the nucleotide sequence of SEQ ID NO: 6.

125. An isolated nucleic acid having the nucleotide sequence of SEQ ID NO: 10.

126. An isolated nucleic acid having the nucleotide sequence of SEQ ID NO: 12.

127. An isolated nucleic acid having the nucleotide sequence of SEQ ID NO: 14.

128. An isolated nucleic acid having the nucleotide sequence of SEQ ID NO: 21.

129. An isolated nucleic acid having the nucleotide sequence of SEQ ID NO: 23.

130. An isolated nucleic acid encoding the polypeptide having the amino acid sequence of SEQ ID NO: 2.

131. An isolated nucleic acid encoding the polypeptide having the amino acid sequence of SEQ ID NO: 3.

132. An isolated nucleic acid encoding the polypeptide having the amino acid sequence of SEQ ID NO: 5.

133. An isolated nucleic acid encoding the polypeptide having the amino acid sequence of SEQ ID NO: 7.

134. An isolated nucleic acid encoding the polypeptide having the amino acid sequence of SEQ ID NO: 11.

135. An isolated nucleic acid encoding the polypeptide having the amino acid sequence of SEQ ID NO: 13.

136. An isolated nucleic acid encoding the polypeptide having the amino acid sequence of SEQ ID NO: 15.

137. An isolated nucleic acid encoding the polypeptide having the amino acid sequence of SEQ ID NO: 17.

138. An isolated nucleic acid encoding the polypeptide having the amino acid sequence of SEQ ID NO: 18.

139. An isolated nucleic acid encoding the polypeptide having the amino acid sequence of SEQ ID NO: 19.

140. An isolated nucleic acid encoding the polypeptide having the amino acid sequence of SEQ ID NO: 20.

141. An isolated nucleic acid encoding the polypeptide having the amino acid sequence of SEQ ID NO: 22.

142. An isolated nucleic acid encoding the polypeptide having the amino acid sequence of SEQ ID NO: 24.

143. An isolated polypeptide selected from the group consisting of:
(i) a TSTP encoded by the nucleic acid sequence selected from the group consisting of SEQ ID NOS: 4, 6, 10, 12, 14, 21, and 23;
(ii) a TSTP selected from the group consisting of SEQ ID NOS: 2, 3, 5, 7, 11, 13, 15, 17, 18, 19, 20, 22, and 24;
(iii) a TSTP encoded by a DNA sequence having at least about 75% identity to a sequence selected from the group consisting of SEQ ID NOS: 4, 6, 10, 12, 14, 21, and 23;
(iv) a TSTP having an amino acid sequence that is at least about 75% identical to the amino acid sequence selected from the group consisting of SEQ ID NOS: 2, 3, 5, 7, 11, 13, 15, 17, 18, 19,20,22, and 24;
(v) a variant of a TSTP encoded by a nucleotide sequence selected from the group consisting of SEQ ID NOS: 4, 610, 12, 14, 21, and 23, wherein said variant contains at least one conservative substitution relative to the TSTP encoded by said nucleotide sequence; and
(vi) a variant of a TSTP having an amino acid sequence selected from the group consisting of SEQ ID NOS: 2, 3, 5, 7, 11, 13, 15, 17, 18, 19, 20, 22, and 24, containing at least one conservative substitution.

144. A polypeptide fragment of the polypeptide of claim 143, wherein said fragment comprises at least about 5 to 7 amino acids.

145. The polypeptide fragment of claim 144, wherein said fragment contains a functional domain of a TSTP.

146. The fragment of claim 145, wherein said functional domain interacts with a compound involved in taste activation or modulation.

147. The fragment of claim 145, wherein said functional domain interacts with a second protein involved in taste signal transduction.

148. The fragment of claim 147, wherein said protein involved in taste signal transduction is a G protein subunit.

149. The fragment of claim 147, wherein said protein involved in taste signal transduction is a G Protein-Coupled Receptor.

150. A chimeric or fusion protein comprising at least part of the amino acid sequence of the polypeptide of claim 143, and at least part of a heterologous amino acid sequence.

151. The chimeric or fusion protein of claim 150, wherein said heterologous sequence is a sequence from a different TSTP.

152. The chimeric or fusion protein of claim 150, wherein said heterologous sequence is a detectable marker gene sequence.

153. A method of screening one or more compounds for the presence of a compound that activates or modulates signal transduction, comprising contacting said one or more compounds with at least about a 5 to 7 amino acid segment of the polypeptide of claim 143.

154. A method for screening one or more proteins for the presence of a protein that interacts with a TSTP expressed selectively in taste receptor cells comprising contacting said one or more proteins with at least about a 5 to 7 amino acid segment of the polypeptide of claim 143.

155. A polypeptide array comprising at least about a 5 to 7 amino acid segment of the polypeptide of claim 143, wherein said polypeptide or polypeptide segment is linked covalently or noncovalently to a solid phase support.

156. An isolated antibody that binds with specificity to the polypeptide of claim 143.

157. An isolated polypeptide having the amino acid sequence of SEQ ID NO: 2.

158. An isolated polypeptide having the amino acid sequence of SEQ ID NO: 3.

159. An isolated polypeptide having the amino acid sequence of SEQ ID NO: 5.

160. An isolated polypeptide having the amino acid sequence of SEQ ID NO: 7.

161. An isolated polypeptide having the amino acid sequence of SEQ ID NO: 11.

162. An isolated polypeptide having the amino acid sequence of SEQ ID NO: 13.

163. An isolated polypeptide having the amino acid sequence of SEQ ID NO: 15.

164. An isolated polypeptide having the amino acid sequence of SEQ ID NO: 17.

165. An isolated polypeptide having the amino acid sequence of SEQ ID NO: 18.

166. An isolated polypeptide having the amino acid sequence of SEQ ID NO: 19.

167. An isolated polypeptide having the amino acid sequence of SEQ ID NO: 20.

168. An isolated polypeptide having the amino acid sequence of SEQ ID NO: 22.

169. An isolated polypeptide having the amino acid sequence of SEQ ID NO: 24.
